(19) United States
(12) Patent Application Publication
(10) Pub. No.: US 2025/0057651 A1
(43) Pub. Date: Feb. 20, 2025

(54) ACTUATION ASSEMBLIES FOR PROSTHETIC HEART VALVES

(71) Applicant: EDWARDS LIFESCIENCES CORPORATION, Irvine, CA (US)

(72) Inventors: Eran Grosu, Kfar-Saba (IL); Michael Bukin, Pardes Hanna (IL); Nikolai Gurovich, Hadera (IL); Anatoly Dvorsky, Haifa (IL)

(21) Appl. No.: 18/938,921

(22) Filed: Nov. 6, 2024

Related U.S. Application Data
(63) Continuation of application No. PCT/US2023/021787, filed on May 10, 2023.
(60) Provisional application No. 63/341,080, filed on May 12, 2022.

(51) Int. Cl. A61F 2/24

(52) U.S. Cl. CPC A61F 2/2418; A61F 2/2439

(57) ABSTRACT

This disclosure is directed to prosthetic heart valves and actuation assemblies for prosthetic heart valves having actuators extending through actuation brackets secured to a prosthetic heart valve frame. In some examples, each actuator extends through and is pivotable relative to two actuation brackets secured to the inner surface of the prosthetic heart valve frame. In other examples, each actuator extends through and is pivotable relative to a vertically extending post of the frame. In this way, the actuators may move angularly relative to the longitudinal axis of the frame during the radial expansion and compression of the prosthetic heart valve. This may prevent bending and buckling of the actuators during the deployment of the prosthetic heart valve.

### CROSS REFERENCE TO RELATED APPLICATIONS

This application is a continuation of PCT Application No. PCT/US2023/021787, filed on May 10, 2023, which claims the benefit of U.S. Provisional Patent Application No. 63/341,080, filed on May 12, 2022, entitled “ACTUATION ASSEMBLIES FOR PROSTHETIC HEART VALVES,” each of which is incorporated by reference herein in its entirety.

### FIELD

The present disclosure relates to implantable expandable prosthetic heart valves and actuation mechanisms for use with expandable prosthetic heart valves.

### BACKGROUND

The human heart can suffer from various valvular diseases. These valvular diseases can result in significant malfunctioning of the heart and ultimately require repair of the native valve or replacement of the native valve with an artificial valve. There are a number of known repair devices (for example, stents) and artificial valves, as well as a number of known methods of implanting these devices and valves in humans. Percutaneous and minimally-invasive surgical approaches are used in various procedures to deliver prosthetic medical devices to locations inside the body that are not readily accessible by surgery or where access without surgery is desirable. In one specific example, a prosthetic heart valve can be mounted in a crimped state on the distal end of a delivery apparatus and advanced through the patient's vasculature (for example, through a femoral artery and the aorta) until the prosthetic heart valve reaches the implantation site in the heart. The prosthetic heart valve is then expanded to its functional size, for example, by inflating a balloon on which the prosthetic valve is mounted, actuating a mechanical actuator that applies an expansion force to the prosthetic heart valve, or by deploying the prosthetic heart valve from a delivery capsule of the delivery apparatus so that the prosthetic heart valve can self-expand to its functional size.

During radial compression of a frame that utilizes mechanical actuators, certain components of the frame and/or the actuators can be subject to varying compressive and/or bending stresses as different portions of the frame compress to varying degrees. These stresses can cause undesirable deformation of the actuators, which can interfere with the deployment of the prosthetic heart valve.

Accordingly, there is a need for frames for prosthetic heart valves that isolate the actuators from the bending stresses caused by radially compressing a prosthetic heart valve.

### SUMMARY

Disclosed herein are prosthetic heart valve assemblies having actuators decoupled from the bending forces caused by the radial compression of the frame of the prosthetic heart valve assemblies. The disclosed prosthetic heart valves can include actuation assemblies mounted to brackets projecting radially inward or outward from the frame and configured to allow the actuators to pivot relative to the frame as the prosthetic heart valves radially expand or radially contract. In other examples, the actuation assemblies can be mounted to a portion of the frame that functions as a pivot joint or hinge and allows the actuator to pivot relative to a longitudinal axis of the frame. Such actuation assemblies can relieve or remove the bending stresses on the actuator, and allow the prosthetic heart valves disclosed herein to be radially compressed without causing the actuators to buckle or deform.

A prosthetic heart valve can comprise a frame and a valvular structure coupled to the frame. In addition to these components, a prosthetic heart valve can further comprise one or more of the components disclosed herein.

In some examples, a prosthetic heart valve can comprise a sealing member configured to reduce paravalvular leakage.

In some examples, a prosthetic heart valve comprises an actuation assembly comprising a first actuation bracket and a second actuation bracket coupled to the frame at axially spaced apart locations.

In some examples, a prosthetic heart valve comprises an actuator extending axially through the first actuation bracket and the second actuation bracket.

In some examples, the first actuation bracket comprises a first chamber.

In some examples, the first chamber contains an internally threaded nut that receives an externally threaded portion of the actuator.

In some examples, an outer diameter of the nut is smaller than an inner diameter of the first chamber, and the axial length of the nut is shorter than the axial length of the first chamber.

In some examples, the second actuation bracket comprises a second chamber.

In some examples, the second chamber contains a sleeve with a smooth internal bore.

In some examples, the sleeve has an outer diameter that is smaller than the inner diameter of the second chamber and an axial length that is shorter than the axial length of the second chamber.

In some examples, the first actuation bracket comprises a ball joint comprising a socket and a sleeve mounted in the socket.

In some examples, the second actuation bracket comprises a ball joint comprising a socket and a sleeve mounted in the socket.

In some examples, a prosthetic heart valve comprises a vertically oriented first post parallel to a longitudinal axis of the frame.

In some examples, a prosthetic heart valve comprises an actuation assembly comprising an actuator operatively coupled to the vertically oriented first post.

In some examples, the vertically oriented first post comprises a channel that receives an end portion of the actuator.

In some examples, the actuator can pivot radially relative to the longitudinal axis of the frame such that a first angle between the actuator and the first post can change when the prosthetic valve moves from a radially compressed configuration to a radially expanded configuration.

In some examples, the actuation assembly decouples bending forces of the frame from the actuator.

In some examples, the actuation assembly allows the frame to bend radially outwards independent of the actuator when the frame moves between a radially expanded configuration and a radially compressed configuration.

Certain examples concern a prosthetic valve, comprising a radially expandable annular frame. The frame has a first end portion, a second end portion, and a longitudinal axis extending between the first end portion and the second end portion and is radially expandable between a radially compressed state and a radially expanded state. The prosthetic heart valve comprises a valvular structure disposed within the frame and configured to regulate the flow of blood through the frame in one direction. The prosthetic heart valve also comprises an actuation assembly comprising a first actuation bracket and a second actuation bracket coupled to the frame at axially spaced apart locations and an actuator extending axially through the first actuation bracket and the second actuation bracket. The actuator is rotatable relative to the first actuation bracket and the second actuation bracket to radially expand or radially compress the frame, and the actuation assembly decouples bending forces of the frame from the actuator, such that the frame can bend radially outwards from the longitudinal axis independent of the actuator when the frame moves between the radially expanded state and the radially compressed state.

Certain examples concern a prosthetic valve, comprising a radially expandable frame. A valvular structure is disposed within the frame and configured to regulate the flow of blood through the frame in one direction, and an actuation assembly operatively coupled to the frame. The actuation assembly comprises a first actuation bracket coupled to the frame at a first location, a second actuation bracket coupled to the frame at a second location axially spaced from the first location, an internally threaded nut disposed within the first actuation bracket, and an actuator extending through the nut and from the first actuation bracket to the second actuation bracket. Rotating the actuator in a first direction relative to the first and second actuation brackets causes the radial expansion and compression of the frame. The actuation assembly decouples bending forces of the frame from the actuator, such that the frame can bend radially outwards from a longitudinal axis of the frame independent of the actuator upon rotation of the actuator.

Certain examples concern a prosthetic valve, comprising a radially expandable frame having a first end portion, a second end portion, and a longitudinal axis extending between the first end portion and the second end portion. The prosthetic heart valve also comprises a vertically oriented first post parallel to the longitudinal axis, a valvular structure disposed within the frame configured to permit the flow of blood from the first end portion of the frame towards the second end portion of the frame and to prohibit the flow of blood from the second end portion of the frame towards the first end portion of the frame, and an actuation assembly comprising an actuator operatively coupled to the first post. Rotating the actuator in a first direction relative to the first post causes the radial expansion of the prosthetic valve, and the actuator can rotate radially relative to the longitudinal axis such that a first angle between the actuator and the first post can change when the prosthetic valve moves from a radially compressed configuration to a radially expanded configuration or from the radially expanded configuration to the radially compressed configuration.

Certain examples concern a prosthetic heart valve, comprising a radially expandable frame having an inflow end, an outflow end, and a longitudinal axis extending from the inflow end to the outflow end. The prosthetic heart valve also comprises a valvular structure disposed within the frame and configured to allow the flow of blood through the frame from the inflow end to the outflow end and prevent the flow of blood from the outflow end towards the inflow end, and an actuation assembly operatively coupled to the frame. The actuation assembly comprises a first actuation bracket positioned towards the inflow end of the frame, a second actuation bracket positioned towards the outflow end of the frame, a nut disposed within the second actuation bracket, and an actuator extending from the first actuation bracket to the second actuation bracket and extending through the nut. Rotating the actuator in a first direction relative to the first and second actuation brackets causes radial expansion of the frame, and rotating the actuator in a second direction relative to the actuation brackets causes radial compression of the frame. The first and second actuation brackets are configured to pivot relative to the longitudinal axis and the actuator upon rotation of the actuator.

Certain examples concern a prosthetic heart valve, comprising a radially expandable frame with a first end portion, a second end portion, a longitudinal axis extending between the first end portion and the second end portion, a vertically oriented first post, and a vertically oriented second post. The prosthetic heart valve also comprises a valvular structure disposed within the frame and configured to regulate the flow of blood through the frame in one direction, and an actuation assembly comprising an actuator operatively coupled to the frame. The vertically oriented first post comprises a fixed end and a free end, having a nut chamber containing a nut configured to tilt relative to the longitudinal axis within the nut chamber and a slot configured to receive an end portion of the actuator. The actuator extends through the nut and is configured to rotate relative to the longitudinal axis to produce radial expansion of the frame from a radially compressed configuration to a radially expanded configuration or radially compression of the frame from the radially expanded configuration to the radially compressed configuration. When the frame is in the radially expanded configuration, the end portion of the actuator is disposed within the slot and when the frame is in the radially compressed configuration, the end portion of the actuator can extend radially outwards from the slot.

Certain examples concern a prosthetic valve, comprising a radially expandable annular frame having a first end portion, a second end portion, and a longitudinal axis extending between the first end portion and the second end portion. The frame is radially expandable between a radially compressed state and a radially expanded state. The prosthetic valve also comprises a valvular structure disposed within the frame and configured to regulate the flow of blood through the frame in one direction and an actuation assembly comprising a first mount, a second mount coupled to the frame at axially spaced apart locations, and an actuator extending axially through the first mount and the second mount. The first mount and second mount each comprise a fixed housing and a sleeve disposed within the fixed housing to form a ball and socket joint, and the sleeve is rotatable relative to the fixed housing and the frame; and wherein the actuator is rotatable relative to the first mount and the second mount to radially expand or radially compress the frame.

In some examples, a prosthetic heart valve comprises one or more of the components recited in Examples 1-75 below.

The various innovations of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the disclosure will become more apparent from the following detailed description, claims, and accompanying figures.

### BRIEF DESCRIPTION OF THE DRAWINGS

FIG. 1A is a perspective view of one example of a prosthetic valve including a frame and a plurality of leaflets attached to the frame.

FIG. 1B is a perspective view of the prosthetic valve of FIG. 1A with an outer skirt disposed around the frame.

FIG. 2A is a perspective view of a frame for the prosthetic valve of FIG. 1A.

FIG. 2B is a front portion of the frame shown in FIG. 2A.

FIG. 3 is a side elevation view of a delivery apparatus for a prosthetic device, such as a prosthetic valve, according to one example.

FIG. 4 is a perspective view of a portion of an actuator of the prosthetic device of FIGS. 1-2 and an actuator assembly of a delivery apparatus, according to one example.

FIG. 5 is a perspective view of the actuator and actuator assembly of FIG. 4 with the actuator assembly physically coupled to the actuator.

FIG. 6 is a schematic, side elevation view of a portion of one example of a frame and an actuation assembly attached to the inner surface of the frame.

FIG. 7A is a schematic, perspective view of the frame section and actuation assembly of FIG. 6.

FIG. 7B is an enlarged, cross-sectional view of a proximal actuation bracket of the actuation assembly shown in FIG. 7A.

FIG. 7C is an enlarged, cross-sectional view of a distal actuation bracket of the actuation assembly shown in FIG. 7A.

FIG. 7D is a perspective view of an actuation bracket according to the example shown in FIGS. 7B and 7C.

FIG. 7E is a perspective view of an actuation bracket according to some examples.

FIG. 8 is a perspective view of a portion of a frame section having an actuator, according to one example.

FIG. 9A is a cut away side view of the proximal end portion of the vertical post of FIG. 8.

FIG. 9B is a side elevation view of the proximal end portion of the vertical post of FIG. 8.

### DETAILED DESCRIPTION

### General Considerations

For purposes of this description, certain aspects, advantages, and novel features of examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present, or problems be solved.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

As used in this application and in the claims, the singular forms “a,” “an,” and “the” include the plural forms unless the context clearly dictates otherwise. Additionally, the term “includes” means “comprises.” Further, the term “coupled” generally means physically, mechanically, chemically, magnetically, and/or electrically coupled or linked and does not exclude the presence of intermediate elements between the coupled or associated items absent specific contrary language.

As used herein, the term “proximal” refers to a position, direction, or portion of a device that is closer to the user and further away from the implantation site. As used herein, the term “distal” refers to a position, direction, or portion of a device that is further away from the user and closer to the implantation site. Thus, for example, proximal motion of a device is motion of the device away from the implantation site and toward the user (for example, out of the patient's body), while distal motion of the device is motion of the device away from the user and toward the implantation site (for example, into the patient's body). The terms “longitudinal” and “axial” refer to an axis extending in the proximal and distal directions, unless otherwise expressly defined.

### Introduction to the Disclosed Technology

Prosthetic valves disclosed herein can be radially compressible and expandable between a radially compressed state and a radially expanded state. Thus, the prosthetic valves can be crimped on or retained by an implant delivery apparatus in the radially compressed state while being advanced through a patient's vasculature on the delivery apparatus. The prosthetic valve can be expanded to the radially expanded state once the prosthetic valve reaches the implantation site. It is understood that the prosthetic valves disclosed herein may be used with a variety of implant delivery apparatuses and can be implanted via various delivery procedures, examples of which will be discussed in more detail later.

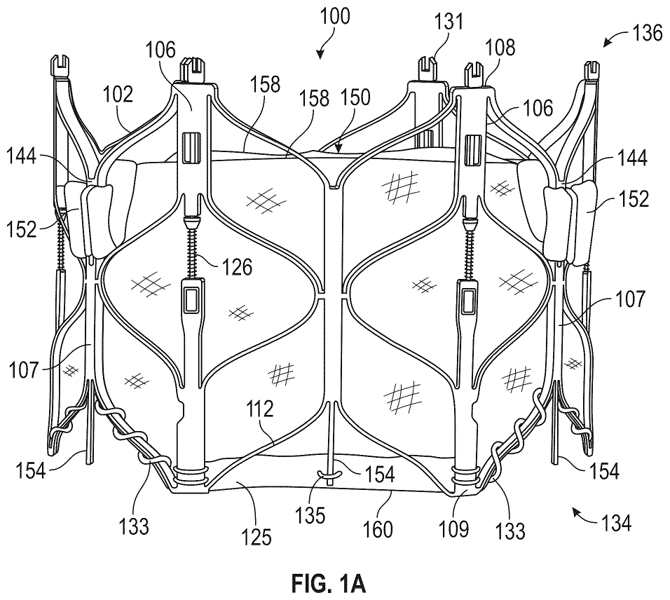

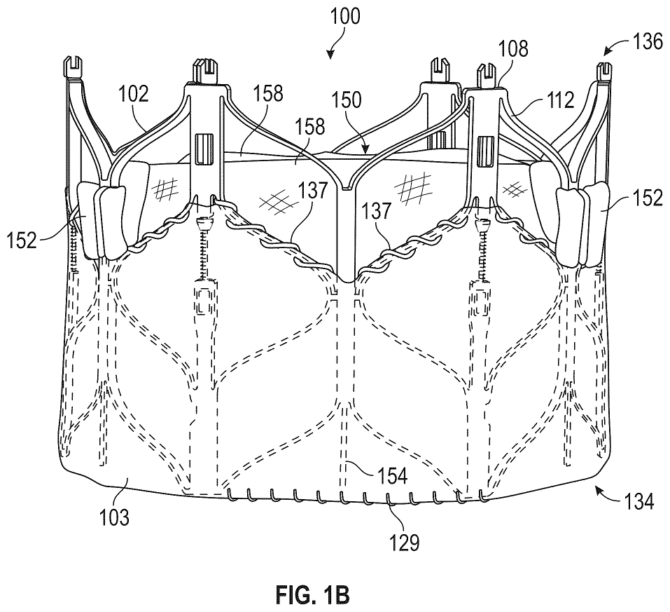

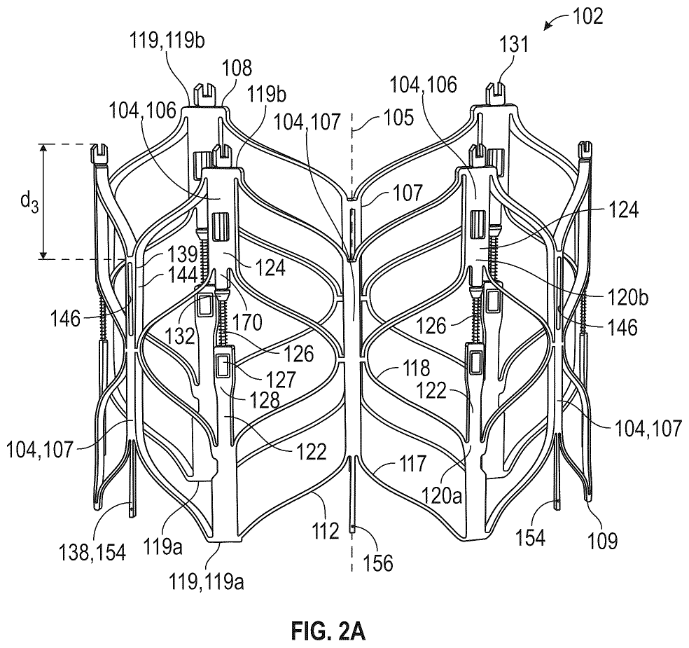

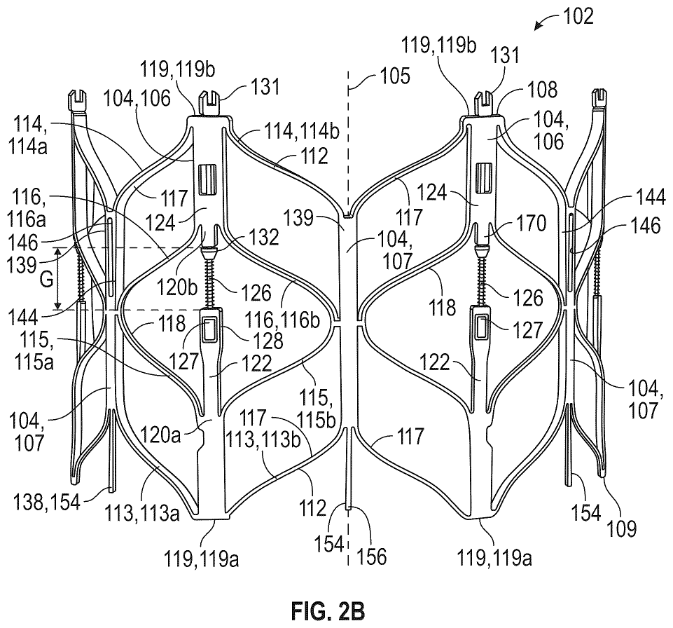

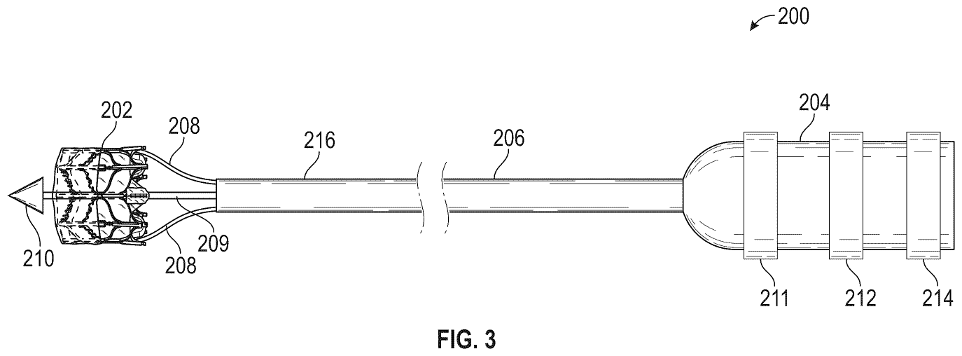

FIGS. 1A-2B illustrate an exemplary prosthetic device (for example, a prosthetic heart valve) that can be advanced through a patient's vasculature, such as to a native heart valve, by a delivery apparatus, such as the exemplary delivery apparatus shown in FIG. 3. The frame of the prosthetic heart valve can include one or more mechanical expansion and locking mechanisms that can be integrated into the frame—specifically, into axially extending posts of the frame. The mechanical expansion and/or locking mechanisms can be removably coupled to, and/or actuated by, the delivery apparatus to radially expand the prosthetic heart valve and lock the prosthetic heart valve in one or more radially expanded states.

### The Disclosed Technology and Examples

FIGS. 1A-2B show an exemplary prosthetic valve 100, according to one example. Any of the prosthetic valves disclosed herein are adapted to be implanted in the native aortic annulus, although in other examples they can be adapted to be implanted in the other native annuluses of the heart (the pulmonary, mitral, and tricuspid valves). The disclosed prosthetic valves also can be implanted within vessels communicating with the heart, including a pulmonary artery (for replacing the function of a diseased pulmonary valve, or the superior vena cava or the inferior vena cava (for replacing the function of a diseased tricuspid valve) or various other veins, arteries and vessels of a patient. The disclosed prosthetic valves also can be implanted within a previously implanted prosthetic valve (which can be a prosthetic surgical valve or a prosthetic transcatheter heart valve) in a valve-in-valve procedure.

In some examples, the disclosed prosthetic valves can be implanted within a docking or anchoring device that is implanted within a native heart valve or a vessel. For instance, in one example, the disclosed prosthetic valves can be implanted within a docking device implanted within the pulmonary artery for replacing the function of a diseased pulmonary valve, such as disclosed in U.S. Publication No. 2017/0231756, which is incorporated by reference herein. In some examples, the disclosed prosthetic valves can be implanted within a docking device implanted within or at the native mitral valve, such as disclosed in PCT Publication No. WO2020/247907, which is incorporated herein by reference. In some examples, the disclosed prosthetic valves can be implanted within a docking device implanted within the superior or inferior vena cava for replacing the function of a diseased tricuspid valve, such as disclosed in U.S. Publication No. 2019/0000615, which is incorporated herein by reference.

FIGS. 1A-2B illustrate an example of a prosthetic valve 100 (which also may be referred to herein as “prosthetic heart valve 100”) having a frame 102. FIGS. 2A-2B show the frame 102 by itself, while FIGS. 1A-1B show the frame 102 with a valvular structure 150 (which can comprise leaflets 158, as described further below) mounted within and to the annular frame 102. FIG. 1B additionally shows an optional skirt assembly comprising an outer skirt 103. While only one side of the frame 102 is depicted in FIG. 2B, it should be appreciated that the frame 102 forms an annular structure having an opposite side that is substantially identical to the portion shown in FIG. 1B, as shown in FIGS. 1A-2A.

As shown in FIGS. 1A and 1B, the valvular structure 150 is coupled to and supported inside the frame 102. The valvular structure 150 is configured to regulate the flow of blood through the prosthetic valve 100, from an inflow end portion 134 to an outflow end portion 136. The valvular structure 150 can include, for example, a leaflet assembly comprising one or more leaflets 158 made of flexible material. The leaflets 158 can be made from in whole or part, biological material, bio-compatible synthetic materials, or other such materials. Suitable biological material can include, for example, bovine pericardium (or pericardium from other sources). The leaflets 158 can be secured to one another at their adjacent sides to form commissures 152, each of which can be secured to a respective commissure support structure 144 (also referred to herein as “commissure supports”) and/or to other portions of the frame 102, as described in greater detail below.

In the example depicted in FIGS. 1A and 1B, the valvular structure 150 includes three leaflets 158, which can be arranged to collapse in a tricuspid arrangement. Each leaflet 158 can have an inflow edge portion 160 (which can also be referred to as a cusp edge portion) (FIG. 1A). The inflow edge portions 160 of the leaflets 158 can define an undulating, curved scallop edge that generally follows or tracks portions of struts 112 of the frame 102 in a circumferential direction when the frame 102 is in the radially expanded configuration. The inflow edge portions 160 of the leaflets 158 can be referred to as a “scallop line.”

The prosthetic valve 100 may include one or more skirts mounted around the frame 102. For example, as shown in FIG. 1B, the prosthetic valve 100 may include an outer skirt 103 mounted around an outer surface of the frame 102. The outer skirt 103 can function as a sealing member for the prosthetic valve 100 by sealing against the tissue of the native valve annulus and helping to reduce paravalvular leakage past the prosthetic valve 100. In some cases, an inner skirt (not shown) may be mounted around an inner surface of the frame 102. The inner skirt can function as a sealing member to prevent or decrease perivalvular leakage, to anchor the leaflets 158 to the frame 102, and/or to protect the leaflets 158 against damage caused by contact with the frame 102 during crimping and during working cycles of the prosthetic valve 100. In some examples, the inflow edge portions 160 of the leaflets 158 can be sutured to the inner skirt generally along the scallop line. The inner skirt can in turn be sutured to adjacent struts 112 of the frame 102. In other examples, as shown in FIG. 1A, the leaflets 158 can be sutured directly to the frame 102 or to a reinforcing member 125 (also referred to as a reinforcing skirt or connecting skirt) in the form of a strip of material (for example, a fabric strip) which is then sutured to the frame 102, along the scallop line via stitches (for example, whip stitches) 133.

The inner and outer skirts and the connecting skirt 125 can be formed from any of various suitable biocompatible materials, including any of various synthetic materials, including fabrics (for example, polyethylene terephthalate fabric) or natural tissue (for example, pericardial tissue). The skirt can be wholly or partly formed of any suitable biological material, synthetic material (for example, any of various polymers), or combinations thereof. In some examples, the skirt can comprise a fabric having interlaced yarns or fibers, such as in the form of a woven, braided, or knitted fabric. In some examples, the fabric can have a plush nap or pile. Exemplary fabrics having a plus nap or pile include velour, velvet, velveteen, corduroy, terrycloth, fleece, etc. In some examples, the skirt can comprise a fabric without interlaced yarns or fibers, such as felt or an electrospun fabric. Exemplary materials that can be used for forming such fabrics (with or without interlaced yarns or fibers) include, without limitation, polyethylene (PET), ultra-high molecular weight polyethylene (UHMWPE), polytetrafluoroethylene (PTFE), expanded polytetrafluoroethylene (ePTFE), polyamide etc. In some examples, the skirt can comprise a non-textile or non-fabric material, such as a film made from any of a variety of polymeric materials, such as PTFE, PET, polypropylene, polyamide, polyetheretherketone (PEEK), polyurethane (such as thermoplastic polyurethane (TPU)), etc. In some examples, the skirt can comprise a sponge material or foam, such as polyurethane foam. In some examples, the skirt can comprise natural tissue, such as pericardium (for example, bovine pericardium, porcine pericardium, equine pericardium, or pericardium from other sources).

Further details regarding the use of skirts or sealing members in prosthetic valves can be found, for example, in U.S. Patent Publication No. 2020/0352711, which is incorporated herein by reference.

Further details regarding the assembly of the leaflet assembly and the assembly of the leaflets and the skirts to the frame can be found, for example, in U.S. Provisional Application Nos. 63/209,904, filed Jun. 11, 2021, and 63/224,534, filed Jul. 22, 2021, which are incorporated herein by reference. Further details of the construction and function of the frame 102 can be found in International Patent Application No. PCT/US2021/052745, filed Sep. 30, 2021, which is incorporated herein by reference.

The frame 102, which is shown alone and in greater detail in FIGS. 2A and 2B, comprises an inflow end 109, an outflow end 108, and a plurality of axially extending posts 104. The axial direction of the frame 102 is indicated by a longitudinal axis 105, which extends from the inflow end 109 to the outflow end 108 (FIGS. 2A and 2B). Some of the posts 104 can be arranged in pairs of axially aligned first and second struts or posts 122, 124. An actuator 126 (such as the illustrated threaded rod or bolt) can extend through one or more pairs of posts 122, 124 to form an integral expansion and locking mechanism or actuator mechanism 106 configured to radially expand and compress the frame 102, as further described below. One or more of posts 104 can be configured as support posts 107.

The actuator mechanisms 106 (which can be used to radially expand and/or radially compress the prosthetic valve 100) can be integrated into the frame 102 of the prosthetic valve 100, thereby reducing the crimp profile and/or bulk of the prosthetic valve 100. Integrating the actuator mechanisms 106 (which can also be referred to herein as “expansion and locking mechanisms”) into the frame 102 can also simplify the design of the prosthetic valve 100, making the prosthetic valve 100 less costly and/or easier to manufacture. In the illustrated example, an actuator 126 extends through each pair of axially aligned posts 122, 124. In other examples, one or more of the pairs of posts 122, 124 can be without a corresponding actuator.

The posts 104 can be coupled together by a plurality of circumferentially extending link members or struts 112. Each strut 112 extends circumferentially between adjacent posts 104 to connect all of the axially extending posts 104. As one example, the prosthetic valve 100 can include equal numbers of support posts 107 and pairs of actuator posts 122, 124, and the pairs of posts 122, 124 and the support posts 107 can be arranged in an alternating order such that each strut 112 is positioned between one of the pairs of posts 122, 124 and one of the support posts 107 (that is, each strut 112 can be coupled on one end to one of the posts 122, 124 and can be coupled on the other end to one of the support posts 107). However, the prosthetic valve 100 can include different numbers of support posts 107 and pairs of posts 122, 124 and/or the pairs of posts 122, 124 and the support posts 107 can be arranged in a non-alternating order, in other examples.

As illustrated in FIG. 2B, the struts 112 can include a first row of struts 113 at or near the inflow end 109 of the prosthetic valve 100, a second row of struts 114 at or near the outflow end 108 of the prosthetic valve 100, and third and fourth rows of struts 115, 116, respectively, positioned axially between the first and second rows of struts 113, 114. The struts 112 can form and/or define a plurality of cells (that is, openings) in the frame 102. For example, the struts 113, 114, 115, and 116 can at least partially form and/or define a plurality of first cells 117 and a plurality of second cells 118 that extend circumferentially around the frame 102. Specifically, each first cell 117 can be formed by two struts 113a, 113b of the first row of struts 113, two struts 114a, 114b of the second row of struts 114, and two of the support posts 107. Each second cell 118 can be formed by two struts 115a, 115b of the third row of struts 115 and two struts 116a, 116b of the fourth row of struts 116. As illustrated in FIGS. 2A and 2B, each second cell 118 can be disposed within one of the first cells 117 (that is, the struts 115a-116b forming the second cells 118 are disposed between the struts forming the first cells 117 (that is, the struts 113a, 113b and the struts 114a, 114b), closer to an axial midline of the frame 102 than the struts 113a-114b).

As illustrated in FIGS. 2A and 2B, the struts 112 of frame 102 can comprise a curved shape. Each first cell 117 can have an axially-extending hexagonal shape including first and second apices 119 (for example, an inflow apex 119a and an outflow apex 119b). In examples where the delivery apparatus is releasably connected to the outflow apices 119b (as described below), each inflow apex 119a can be referred to as a “distal apex” and each outflow apex 119b can be referred to as a “proximal apex”. Each second cell 118 can have a diamond shape including first and second apices 120 (for example, distal apex 120a and proximal apex 120b). In some examples, the frame 102 comprises six first cells 117 extending circumferentially in a row, six second cells 118 extending circumferentially in a row within the six first cells 117, and twelve posts 104. However, in other examples, the frame 102 can comprise a greater or fewer number of first cells 117 and a correspondingly greater or fewer number of second cells 118 and posts 104.

As noted above, some of the posts 104 can be arranged in pairs of first and second posts 122, 124. The posts 122, 124 are aligned with each other along the length of the frame 102 and are axially separated from one another by a gap G (FIG. 2B) (those with actuators 126 can be referred to as actuator posts or actuator struts). Each first post 122 (that is, the lower post shown in FIGS. 2A and 2B) can extend axially from the inflow end 109 of the prosthetic valve 100 toward the second post 124, and the second post 124 (that is, the upper post shown in FIGS. 2A and 2B) can extend axially from the outflow end 108 of the prosthetic valve 100 toward the first post 122. For example, each first post 122 can be connected to and extend from an inflow apex 119a and each second post 124 can be connected to and extend from an outflow apex 119b. Each first post 122 and the second post 124 can include an inner bore configured to receive a portion of an actuator member, such as in the form of a substantially straight threaded rod 126 (or bolt) as shown in the illustrated example. The threaded rod 126 also may be referred to herein as actuator 126, actuator member 126, and/or screw actuator 126. In examples where the delivery apparatus can be releasably connected to the outflow end 108 of the frame 102, the first posts 122 can be referred to as distal posts or distal axial struts and the second posts 124 can be referred to as proximal posts or proximal axial struts.

Each threaded rod 126 extends axially through a corresponding first post 122 and second post 124. Each threaded rod 126 also extends through a bore of a nut 127 captured within a slot or window formed in an end portion 128 of the first post 122. The threaded rod 126 has external threads that engage internal threads of the bore of the nut 127. The inner bore of the second post 124 (through which the threaded rod 126 extends) can have a smooth and/or non-threaded inner surface to allow the threaded rod 126 to slide freely within the bore. Rotation of the threaded rod 126 relative to the nut 127 produces radial expansion and compression of the frame 102, as further described below.

In some examples, the threaded rod 126 can extend past the nut 127 toward the inflow end 109 of the frame 102 into the inner bore of the first post 122. The nut 127 can be held in a fixed position relative to the first post 122 such that the nut 127 does not rotate relative to the first post 122. In this way, whenever the threaded rod 126 is rotated (for example, by a physician) the threaded rod 126 can rotate relative to both the nut 127 and the first post 122. The engagement of the external threads of the threaded rod 126 and the internal threads of the nut 127 prevent the rod 126 from moving axially relative to the nut 127 and the first post 122 unless the threaded rod 126 is rotated relative to the nut 127. Thus, the threaded rod 126 can be retained or held by the nut 127 and can only be moved relative to the nut 127 and/or the first post 122 by rotating the threaded rod 126 relative to the nut 127 and/or the first post 122. In other examples, in lieu of using the nut 127, at least a portion of the inner bore of the first post 122 can be threaded. For example, the bore along the end portion 128 of the first post 122 can comprise inner threads that engage the external threaded rod 126 such that rotation of the threaded rod causes the threaded rod 126 to move axially relative to the first post 122.

When a threaded rod 126 extends through and/or is otherwise coupled to a pair of axially aligned posts 122, 124, the pair of axially aligned posts 122, 124 and the threaded rod 126 can serve as one of the expansion and locking mechanisms 106. In some examples, a threaded rod 126 can extend through each pair of axially aligned posts 122, 124 so that all of the posts 122, 124 (with their corresponding rods 126) serve as expansion and locking mechanisms 106. As just one example, the prosthetic valve 100 can include six pairs of posts 122, 124, and each of the six pairs of posts 122, 124 with their corresponding rods 126 can be configured as one of the expansion and locking mechanisms 106 for a total of six expansion and locking mechanisms 106. In other examples, not all pairs of posts 122, 124 need be expansion and locking mechanisms (that is, actuators). If a pair of posts 122, 124 is not used as an expansion and locking mechanism, a threaded rod 126 need not extend through the posts 122, 124 of that pair.

The threaded rod 126 can be rotated relative to the nut 127, the first post 122, and the second post 124 to axially foreshorten and/or axially elongate the frame 102, thereby radially expanding and/or radially compressing, respectively, the frame 102 (and therefore the prosthetic valve 100). Specifically, when the threaded rod 126 is rotated relative to the nut 127, the first post 122, and the second post 124, the first and second posts 122, 124 can move axially relative to one another, thereby widening or narrowing the gap G (FIG. 2B) separating the posts 122, 124, and thereby radially compressing or radially expanding the prosthetic valve 100, respectively. Thus, the gap G (FIG. 2B) between the first and second posts 122, 124 narrows as the frame 102 is radially expanded and widens as the frame 102 is radially compressed.

The threaded rod 126 can extend proximally past the proximal end of the second post 124 and can include a head portion 131 at its proximal end that can serve at least two functions. First, the head portion 131 can removably or releasably couple the threaded rod 126 to a respective actuator assembly of a delivery apparatus that can be used to radially expand and/or radially compress the prosthetic valve 100 (for example, the delivery apparatus 200 of FIG. 3, as described below). Second, the head portion 131 can prevent the second post 124 from moving proximally relative to the threaded rod 126 and can apply a distally directed force to the second post 124, such as when radially expanding the prosthetic valve 100. Specifically, the head portion 131 can have a width greater than a diameter of the inner bore of the second post 124 such that the head portion 131 is prevented from moving into the inner bore of the second post 124. Thus, as the threaded rod 126 is threaded farther into the nut 127, the head portion 131 of the threaded rod 126 draws closer to the nut 127 and the first post 122, thereby drawing the second post 124 towards the first post 122, and thereby axially foreshortening and radially expanding the prosthetic valve 100.

The threaded rod 126 also can include a stopper 132 (for example, in the form of a nut, washer, or flange) disposed thereon. The stopper 132 can be disposed on the threaded rod 126 such that it sits within the gap G. Further, the stopper 132 can be integrally formed on or fixedly coupled to the threaded rod 126 such that it does not move relative to the threaded rod 126. Thus, the stopper 132 can remain in a fixed axial position on the threaded rod 126 such that it moves in lockstep with the threaded rod 126.

Rotation of the threaded rod 126 in a first direction (for example, clockwise) can cause corresponding axial movement of the first and second posts 122, 124 toward one another, thereby decreasing the gap G and radially expanding the frame 102, while rotation of the threaded rod 126 in an opposite second direction causes corresponding axial movement of the first and second posts 122, 124 away from one another, thereby increasing the gap G and radially compressing the frame 102. When the threaded rod 126 is rotated in the first direction, the head portion 131 of the rod 126 bears against an adjacent surface of the frame (for example, an outflow apex 119b), while the nut 127 and the first post 122 travel proximally along the threaded rod 126 toward the second post 124, thereby radially expanding the frame 102. As the frame 102 moves from a compressed configuration to an expanded configuration, the gap G between the first and second posts 122, 124 can narrow.

When the threaded rod 126 is rotated in the second direction, the threaded rod 126 and the stopper 132 move toward the outflow end 108 of the frame 102 until the stopper 132 abuts the inflow end 170 of the second post 124 (as shown in FIGS. 2A and 2B). Upon further rotation of the rod 126 in the second direction, the stopper 132 can apply a proximally directed force to the second post 124 to radially compress the frame 102. Specifically, during crimping/radial compression of the prosthetic valve 100, the threaded rod 126 can be rotated in the second direction (for example, counterclockwise) causing the stopper 132 to push against (that is, provide a proximally directed force to) the inflow end 170 of the second post 124, thereby causing the second post 124 to move away from the first post 122, and thereby axially elongating and radially compressing the prosthetic valve 100.

Thus, each of the second posts 124 can slide axially relative to a corresponding one of the first posts 122 but can be axially retained and/or restrained between the head portion 131 of a threaded rod 126 and a stopper 132. That is, each second post 124 can be restrained at its proximal end by the head portion 131 of the threaded rod 126 and at its distal end by the stopper 132. In this way, the head portion 131 can apply a distally directed force to the second post 124 to radially expand the prosthetic valve 100 while the stopper 132 can apply a proximally directed force to the second post 124 to radially compress the prosthetic valve 100. As explained above, radially expanding the prosthetic valve 100 axially foreshortens the prosthetic valve 100, causing an inflow end portion 134 and outflow end portion 136 of the prosthetic valve 100 (FIGS. 1A and 1B) to move towards one another axially, while radially compressing the prosthetic valve 100 axially elongates the prosthetic valve 100, causing the inflow and outflow end portions 134, 136 to move away from one another axially.

In other examples, the threaded rod 126 can be fixed against axial movement relative to the second post 124 (and the stopper 132 can be omitted) such that rotation of the threaded rod 126 in the first direction produces proximal movement of the nut 127 and radial expansion of the frame 102 and rotation of the threaded rod 126 in the second direction produces distal movement of the nut 127 and radial compression of the frame 102.

As also introduced above, some of the posts 104 can be configured as support posts 107. As shown in FIGS. 2A and 2B, the support posts 107 can extend axially between the inflow and outflow ends 109, 108 of the frame 102 and each can have an inflow end portion 138 and an outflow end portion 139. The outflow end portion 139 of one or more support posts 107 can include a commissure support structure or member 144. The commissure support structure 144 can comprise strut portions defining a commissure opening 146 therein.

The commissure opening 146 (which can also be referred to herein as a “commissure window 146”) can extend radially through a thickness of the support post 107 and can be configured to accept a portion of a valvular structure 150 (for example, a commissure 152) to couple the valvular structure 150 to the frame 102. For example, each commissure 152 can be mounted to a respective commissure support structure 144, such as by inserting a pair of commissure tabs of adjacent leaflets 158 through the commissure opening 146 and suturing the commissure tabs to each other and/or the commissure support structure 144. In some examples, the commissure opening 146 can be fully enclosed by the support post 107 such that a portion of the valvular structure 150 can be slid radially through the commissure opening 146, from an interior to an exterior of the frame 102, during assembly. In the illustrated example, the commissure opening 146 has a substantially rectangular shape that is shaped and sized to receive commissure tabs of two adjacent leaflets therethrough. However, in other examples, the commissure opening can have any of various shapes (for example, square, oval, square-oval, triangular, L-shaped, T-shaped, C-shaped, etc.).

The commissure openings 146 are spaced apart about the circumference of frame 102 (or angularly spaced apart about frame 102). The spacing may or may not be even. In one example, the commissure openings 146 are axially offset from the outflow end 108 of the frame 102 by an offset distance d₃ (indicated in FIG. 2A). As an example, the offset distance d₃ may be in a range from 2 mm to 6 mm. In general, the offset distance d₃ should be selected such that when the leaflets 158 are attached to the frame 102 via the commissure openings 146, the free edge portions (for example, outflow edge portions) of the leaflets 158 will not protrude from or past the outflow end 108 of the frame 102.

The frame 102 can comprise any number of support posts 107, any number of which can be configured as commissure support structures 144. For example, the frame 102 can comprise six support posts 107, three of which are configured as commissure support structures 144. However, in other examples, the frame 102 can comprise more or less than six support posts 107 and/or more or less than three commissure support structures 144.

The inflow end portion 138 of each support post 107 can comprise an extension 154 (show as a cantilevered strut in FIGS. 2A and 2B) that extends toward the inflow end 109 of the frame 102. Each extension 154 can comprise an aperture 156 extending radially through a thickness of the extension 154. In some examples, the extension 154 can extend such that an inflow edge of the extension 154 aligns with or substantially aligns with the inflow end 109 of the frame 102. In use, the extension 154 can prevent or mitigate portions of an outer skirt from extending radially inwardly and thereby prevent or mitigate any obstruction of flow through the frame 102 caused by the outer skirt. The extensions 154 can further serve as supports to which portions of the inner and/or outer skirts and/or the leaflets and/or the connecting skirt 125 can be coupled. For example, sutures used to connect the inner and/or outer skirts and/or the leaflets and/or the connecting skirt 125 can be wrapped around the extensions 154 and/or can extend through apertures 156.

As an example, each extension 154 can have an aperture 156 (FIG. 2A) or other features to receive a suture or other attachment material for connecting an adjacent inflow edge portion 160 of a leaflet 158 (FIG. 1A), the outer skirt 103 (in FIG. 1B), the connecting skirt 125, and/or an inner skirt. In some examples, the inflow edge portion 160 of each leaflet 158 can be connected to a corresponding extension via a suture 135 (FIG. 1A).

In some examples, the outer skirt 103 can be mounted around the outer surface of frame 102 as shown in FIG. 1B and the inflow edge of the outer skirt 103 (lower edge in FIG. 1B) can be attached to the connecting skirt 125 and/or the inflow edge portions 160 of the leaflets 158 that have already been secured to frame 102 as well as to the extensions 154 of the frame 102 by sutures 129. The outflow edge of the outer skirt 103 (the upper edge in FIG. 1B) can be attached to selected struts with stitches 137. In implementations where the prosthetic valve 100 includes an inner skirt, the inflow edge of the inner skirt can be secured to the inflow edge portions 160 before securing the cusp edge portions to the frame 102 so that the inner skirt will be between the leaflets 158 and the inner surface of the frame 102. After the inner skirt and leaflets 158 are secured in place, then the outer skirt 103 can be mounted around the frame 102 as described above.

The frame 102 can be a unitary and/or fastener-free frame that can be constructed from a single piece of material (for example, Nitinol, stainless steel or a cobalt-chromium alloy), such as in the form of a tube. The plurality of cells can be formed by removing portions (for example, via laser cutting) of the single piece of material. The threaded rods 126 can be separately formed and then be inserted through the bores in the second (proximal) posts 124 and threaded into the threaded nuts 127.

In some examples, the frame 102 can be formed from a plastically-expandable material, such as stainless steel or a cobalt-chromium alloy. When the frame 102 is formed from a plastically-expandable material, the prosthetic valve 100 can be placed in a radially compressed state along the distal end portion of a delivery apparatus for insertion into a patient's body. When at the desired implantation site, the frame 102 (and therefore the prosthetic valve 100) can be radially expanded from the radially compressed state to a radially expanded state via actuation of actuation assemblies of the delivery apparatus (as further described below), which rotate the rods 126 to produce expansion of the frame 102. During delivery to the implantation site, the prosthetic valve 100 can be placed inside of a delivery capsule (sheath) to protect against the prosthetic valve 100 contacting the patient's vasculature, such as when the prosthetic valve 100 is advanced through a femoral artery. The capsule can also retain the prosthetic valve 100 in a compressed state having a slightly smaller diameter and crimp profile than may be otherwise possible without a capsule by preventing any recoil (expansion) of the frame 102 once it is crimped onto the delivery apparatus.

In other examples, the frame 102 can be formed from a self-expandable material (for example, Nitinol). When the frame 102 is formed from a self-expandable material, the prosthetic valve 100 can be radially compressed and placed inside the capsule of the delivery apparatus to maintain the prosthetic valve 100 in the radially compressed state while it is being delivered to the implantation site. When at the desired implantation site, the prosthetic valve 100 is deployed or released from the capsule. In some examples, the frame 102 (and therefore the prosthetic valve 100) can partially self-expand from the radially compressed state to a partially radially expanded state. The frame 102 (and therefore the prosthetic valve 100) can be further radially expanded from the partially expanded state to a further radially expanded state via actuation of actuation assemblies of the delivery apparatus (as further described below), which rotate the rods 126 to produce expansion of the frame 102.

The frame 102 can be made of any of various suitable plastically-expandable materials (for example, stainless steel, etc.) or self-expanding materials (for example, Nitinol) as known in the art. When constructed of a plastically-expandable material, the frame 102 (and thus the valve 100) can be crimped to a radially compressed state on a delivery catheter and then expanded inside a patient by an inflatable balloon or equivalent expansion mechanism. When constructed of a self-expandable material, the frame 102 (and thus the prosthetic valve 100) can be crimped to a radially compressed state and restrained in the compressed state by insertion into a sheath or equivalent mechanism of a delivery catheter. Once inside the body, the prosthetic valve 100 can be advanced from the delivery sheath, which allows the prosthetic valve 100 to expand to its functional size.

Suitable plastically-expandable materials that can be used to form the frames disclosed herein (for example, the frame 102) include, metal alloys, polymers, or combinations thereof. Example metal alloys can comprise one or more of the following: nickel, cobalt, chromium, molybdenum, titanium, or other biocompatible metal. In some examples, the frame 102 can comprise stainless steel. In some examples, the frame 102 can comprise cobalt-chromium. In some examples, the frame 102 can comprise nickel-cobalt-chromium. In some examples, the frame 102 comprises a nickel-cobalt-chromium-molybdenum alloy, such as MP35N™ (tradename of SPS Technologies), which is equivalent to UNS R30035 (covered by ASTM F562-02). MP35N™/UNS R30035 comprises 35% nickel, 35% cobalt, 20% chromium, and 10% molybdenum, by weight.

As introduced above, the threaded rods 126 can removably couple the prosthetic valve 100 to actuator assemblies of a delivery apparatus. Referring to FIG. 3, it illustrates an exemplary delivery apparatus 200 for delivering a prosthetic device or valve 202 (for example, prosthetic valve 100) to a desired implantation location. The prosthetic valve 202 can be releasably coupled to the delivery apparatus 200. It should be understood that the delivery apparatus 200 and other delivery apparatuses disclosed herein can be used to implant prosthetic devices other than prosthetic valves, such as stents or grafts.

The delivery apparatus 200 in the illustrated example generally includes a handle 204, a first elongated shaft 206 (which comprises an outer shaft in the illustrated example) extending distally from the handle 204, at least one actuator assembly 208 extending distally through the first shaft 206, a second elongated shaft 209 (which comprises an inner shaft in the illustrated example) extending through the first shaft 206, and a nosecone 210 coupled to a distal end portion of the second shaft 209. The second shaft 209 and the nosecone 210 can define a guidewire lumen for advancing the delivery apparatus through a patient's vasculature over a guidewire. The at least one actuator assembly 208 can be configured to radially expand and/or radially collapse the prosthetic valve 202 when actuated, such as by one or more knobs 211, 212, 214 included on the handle 204 of the delivery apparatus 200.

Though the illustrated example shows two actuator assemblies 208 for purposes of illustration, it should be understood that one actuator assembly 208 can be provided for each actuator (for example, actuator or threaded rod 126) on the prosthetic valve 202. For example, three actuator assemblies 208 can be provided for a prosthetic valve having three actuators. In other examples, a greater or fewer number of actuator assemblies 208 can be present.

In some examples, a distal end portion 216 of the shaft 206 can be sized to house the prosthetic valve 202 in its radially compressed, delivery state during delivery of the prosthetic valve 202 through the patient's vasculature. In this manner, the distal end portion 216 functions as a delivery sheath or capsule for the prosthetic valve 202 during delivery.

The actuator assemblies 208 can be releasably coupled to the prosthetic valve 202. For example, in the illustrated example, each actuator assembly 208 can be coupled to a respective actuator (for example, threaded rod 126) of the prosthetic valve 202. Each actuator assembly 208 can comprise a support tube and an actuator member. When actuated, the actuator assembly can transmit pushing and/or pulling forces to portions of the prosthetic valve 202 to radially expand and collapse the prosthetic valve 202 as previously described. The actuator assemblies 208 can be at least partially disposed radially within, and extend axially through, one or more lumens of the first shaft 206. For example, the actuator assemblies 208 can extend through a central lumen of the shaft 206 or through separate respective lumens formed in the shaft 206.

The handle 204 of the delivery apparatus 200 can include one or more control mechanisms (for example, knobs or other actuating mechanisms) for controlling different components of the delivery apparatus 200 in order to expand and/or deploy the prosthetic valve 202. For example, in the illustrated example the handle 204 comprises first, second, and third knobs 211, 212, and 214, respectively.

The first knob 211 can be a rotatable knob configured to produce axial movement of the first shaft 206 relative to the prosthetic valve 202 in the distal and/or proximal directions in order to deploy the prosthetic valve 202 from the delivery sheath 216 once the prosthetic valve has been advanced to a location at or adjacent the desired implantation location with the patient's body. For example, rotation of the first knob 211 in a first direction (for example, clockwise) can retract the sheath 216 proximally relative to the prosthetic valve 202 and rotation of the first knob 211 in a second direction (for example, counterclockwise) can advance the sheath 216 distally. In other examples, the first knob 211 can be actuated by sliding or moving the first knob 211 axially, such as pulling and/or pushing the knob. In other examples, actuation of the first knob 211 (rotation or sliding movement of the first knob 211) can produce axial movement of the actuator assemblies 208 (and therefore the prosthetic valve 202) relative to the delivery sheath 216 to advance the prosthetic valve 202 distally from the sheath 216.

The second knob 212 can be a rotatable knob configured to produce radial expansion and/or compression of the prosthetic valve 202. For example, rotation of the second knob 212 can rotate the threaded rods 126 of the prosthetic valve 202 via the actuator assemblies 208. Rotation of the second knob 212 in a first direction (for example, clockwise) can radially expand the prosthetic valve 202 and rotation of the second knob 212 in a second direction (for example, counterclockwise) can radially collapse the prosthetic valve 202. In other examples, the second knob 212 can be actuated by sliding or moving the second knob 212 axially, such as pulling and/or pushing the knob.

The third knob 214 can be a rotatable knob operatively connected to a proximal end portion of each actuator assembly 208. The third knob 214 can be configured to retract an outer sleeve or support tube of each actuator assembly 208 to disconnect the actuator assemblies 208 from the proximal portions of the actuators of the prosthetic valve 202 (for example, threaded rod 126). Once the actuator assemblies 208 are uncoupled from the prosthetic valve 202, the delivery apparatus 200 can be removed from the patient, leaving just the prosthetic valve 202 in the patient.

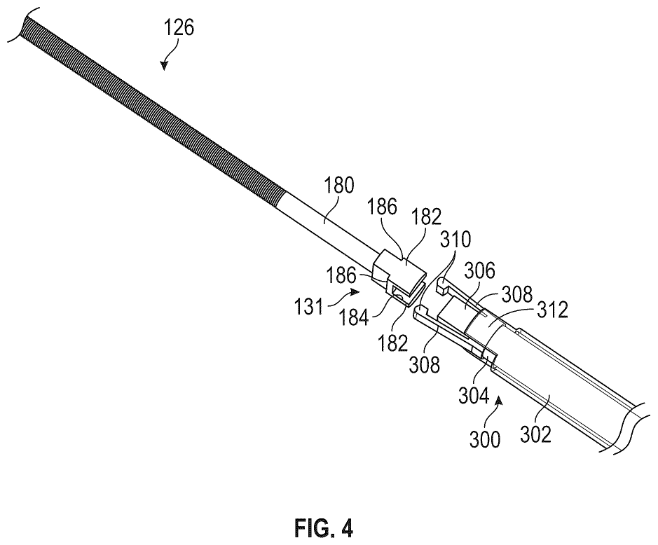

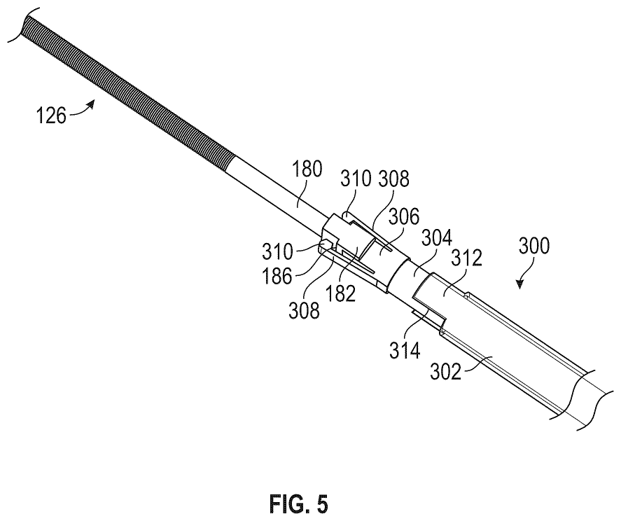

Referring to FIGS. 4-5, they illustrate how each of the threaded rods 126 of the prosthetic valve 100 can be removably coupled to an exemplary actuator assembly 300 (for example, actuator assemblies 208) of a delivery apparatus (for example, delivery apparatus 200). Specifically, FIG. 5 illustrates how one of the threaded rods 126 can be coupled to an actuator assembly 300, while FIG. 4 illustrates how the threaded rod 126 can be detached from the actuator assembly 300.

As introduced above, an actuator assembly 300 can be coupled to the head portion 131 of each threaded rod 126. The head portion 131 can be included at a proximal end portion 180 of the threaded rod 126 and can extend proximally past a proximal end of the second post 124 (FIG. 2A). The head portion 131 can comprise first and second protrusions 182 defining a channel or slot 184 between them, and one or more shoulders 186. As discussed above, the head portion 131 can have a width greater than a diameter of the inner bore of the second post 124 such that the head portion 131 is prevented from moving into the inner bore of the second post 124 and such that the head portion 131 abuts the outflow end 108 of the frame 102. In particular, the head portion 131 can abut an outflow apex 119b of the frame 102. The head portion 131 can be used to apply a distally directed force to the second post 124, for example, during radial expansion of the frame 102.

Each actuator assembly 300 can comprise a first actuation member configured as a support tube or outer sleeve 302 and a second actuation member configured as a driver 304. The driver 304 can extend through the outer sleeve 302. The outer sleeve 302 is shown transparently in FIGS. 4-5 for purposes of illustration. The distal end portions of the outer sleeve 302 and driver 304 can be configured to engage or abut the proximal end of the threaded rod 126 (for example, the head portion 131) and/or the frame 102 (for example, the apex 119b). The proximal portions of the outer sleeve 302 and driver 304 can be operatively coupled to the handle of a delivery apparatus (for example, handle 204). The delivery apparatus in this example can include the same features described previously for delivery apparatus 200. In particular examples, the proximal end portions of each driver 304 can be operatively connected to the knob 212 such that rotation of the knob 212 (clockwise or counterclockwise) causes corresponding rotation of the drivers 304. The proximal end portions of each outer sleeve 302 can be operatively connected to the knob 214 such that rotation of the knob 214 (clockwise or counterclockwise) causes corresponding axial movement of the sleeves 302 (proximally or distally) relative to the drivers 304. In other examples, the handle can include electric motors for actuating these components.

The distal end portion of the driver 304 can comprise a central protrusion 306 configured to extend into the slot 184 of the threaded rod 126, and one or more flexible elongated elements or arms 308 including protrusions or teeth 310 configured to be releasably coupled to the shoulders 186 of the threaded rod 126. The protrusions 310 can extend radially inwardly toward a longitudinal axis of the second actuation member 304. As shown in FIGS. 4-5, the elongated elements 308 can be configured to be biased radially outward to an expanded state, for example, by shape setting the elements 308.

As shown in FIG. 5, to couple the actuator assembly 300 to the threaded rod 126, the driver 304 can be positioned such that the central protrusion 306 is disposed within the slot 184 (FIG. 4) and such that the protrusions 310 of the elongated elements 308 are positioned distally to the shoulders 186. As the outer sleeve 302 is advanced (for example, distally) over the driver 304, the sleeve 302 compresses the elongated elements 308 they abut and/or snap over the shoulders 186, thereby coupling the actuator assembly 300 to the threaded rod 126. Thus, the outer sleeve 302 effectively squeezes and locks the elongated elements 308 and the protrusions 310 of the driver 304 into engagement with (that is, over) the shoulders 186 of the threaded rod 126, thereby coupling the driver 304 to the threaded rod 126.

Because the central protrusion 306 of the driver 304 extends into the slot 184 of the threaded rod 126 when the driver 304 and the threaded rod 126 are coupled, the driver 304 and the threaded rod 126 can be rotational locked such that they co-rotate. So coupled, the driver 304 can be rotated (for example, using knob 212 the handle of the delivery apparatus 200) to cause corresponding rotation of the threaded rod 126 to radially expand or radially compress the prosthetic heart valve 100. The central protrusion 306 can be configured (for example, sized and shaped) such that it is advantageously spaced apart from the inner walls of the outer sleeve 302, such that the central protrusion 306 does not frictionally contact the outer sleeve 302 during rotation. Though in the illustrated example the central protrusion 306 has a substantially rectangular shape in cross-section, in other examples, the protrusion 306 can have any of various shapes, for example, square, triangular, oval, etc. The slot 184 can be correspondingly shaped to receive the protrusion 306.

The outer sleeve 302 can be advanced distally relative to the driver 304 past the elongated elements 308, until the outer sleeve 302 engages the frame 102 (for example, a second post 124 of the frame 102). The distal end portion of the outer sleeve 302 also can comprise first and second support extensions 312 defining gaps or notches 314 between the extensions 312. The support extensions 312 can be oriented such that, when the actuator assembly 300 is coupled to a respective threaded rod 126, the support extensions 312 extend partially over an adjacent end portion (for example, the upper end portion) of one of the second posts 124 on opposite sides of the post 124. The engagement of the support extensions 312 with the frame 102 in this manner can counter-act rotational forces applied to the frame 102 by the rods 126 during expansion of the frame 102. In the absence of a counterforce acting against these rotational forces, the frame 102 can tend to “jerk” or rock in the direction of rotation of the rods 126 when they are actuated to expand the frame 102. The illustrated configuration is advantageous in that outer sleeves, when engaging the proximal posts 124 of the frame 102, can prevent or mitigate such jerking or rocking motion of the frame 102 when the frame 102 is radially expanded.

To decouple the actuator assembly 300 from the prosthetic heart valve 100, the sleeve 302 can be withdrawn proximally relative to the driver 304 until the sleeve 302 no longer covers the elongated elements 308 of the driver 304. As described above, the sleeve 302 can be used to hold the elongated elements 308 against the shoulders 186 of the threaded rod 126 since the elongated elements 308 can be naturally biased to a radial outward position where the elongated elements 308 do not engage the shoulders 186 of the threaded rod 126. Thus, when the sleeve 302 is withdrawn such that it no longer covers/constrains the elongated elements 308, the elongated elements 308 can naturally and/or passively deflect away from, and thereby release from, the shoulders 186 of the threaded rod 126, thereby decoupling the driver 304 from the threaded rod 126.

The sleeve 302 can be advanced (moved distally) and/or retracted (moved proximally) relative to the driver 304 via a control mechanism (for example, knob 214) on the handle 204 of the delivery apparatus 200, by an electric motor, and/or by another suitable actuation mechanism. For example, the physician can turn the knob 214 in a first direction to apply a distally directed force to the sleeve 302 and can turn the knob 214 in an opposite second direction to apply a proximally directed force to the sleeve 302. Thus, when the sleeve 302 does not abut a prosthetic device such as the prosthetic heart valve 100 and the physician rotates the knob 214 in the first direction, the sleeve 302 can move distally relative to the driver 304, thereby advancing the sleeve 302 over the driver 304. When the sleeve 302 does abut the prosthetic device, the physician can rotate the knob 214 in the first direction to push the entire prosthetic device, such as the prosthetic heart valve 100 distally via the sleeve 302. Further, when the physician rotates the knob 214 in the second direction, the sleeve 302 can move proximally relative to the driver 304, thereby withdrawing/retracting the sleeve 302 from the driver 304.

Because the frame is formed (for example, laser cut and optionally shape set) in its radially expanded state, the frame tends to assume a barrel shape as the frame is radially compressed from the radially expanded state to the radially compressed state such that a middle portion of the frame has a slightly larger diameter than the opposing ends of the frame. Further, when the actuator rods are rotated to radially compress the frame, the frame portions coupled to the actuator rods place the actuator rods in a state of axial compression. Because the actuator rods 126 are relatively unsupported (or less supported) along the middle portion of the frame compared the inflow and outflow end portions of the frame, the tendency of the frame to assume a barrel shape in combination with the axial compression forces acting on the actuator rods may cause the actuator rods 126 to buckle or bow outwardly when they are actuated to radially compress the frame, which can result in undesirable deformation of the actuator rods and/or portions of the frame through which the actuator rods extend. Disclosed herein are examples of prosthetic heart valves and actuation mechanisms for prosthetic heart valves having actuation assembly configurations that reduce or prevent the buckling or bending of the actuator rods when the prosthetic valve is radially compressed. In particular, the examples of actuation mechanisms for prosthetic heart valves described below are configured to retain the actuators (for example, threaded rods) in a straight or substantially straight configuration, even if the frame begins to form a barrel shape when radially compressed. In particular examples, the actuation mechanisms can isolate or substantially isolate the actuators from bending forces of the frame.

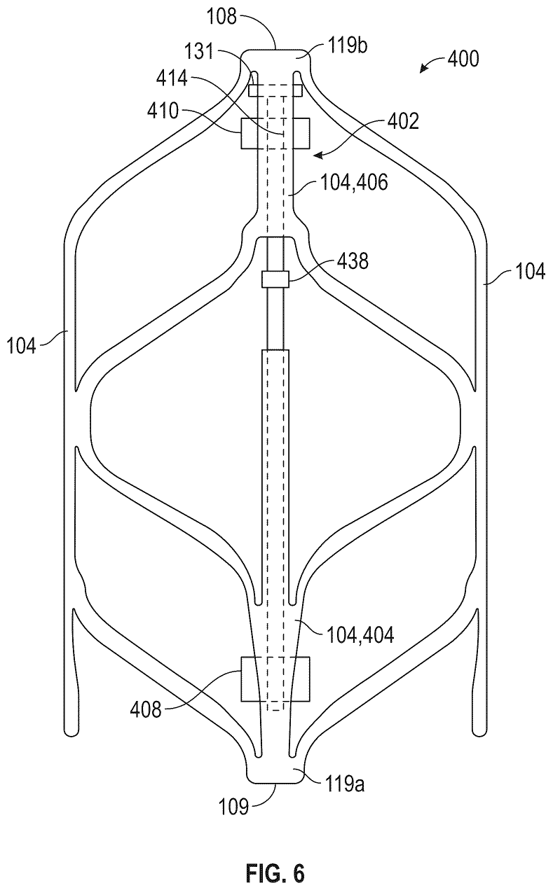

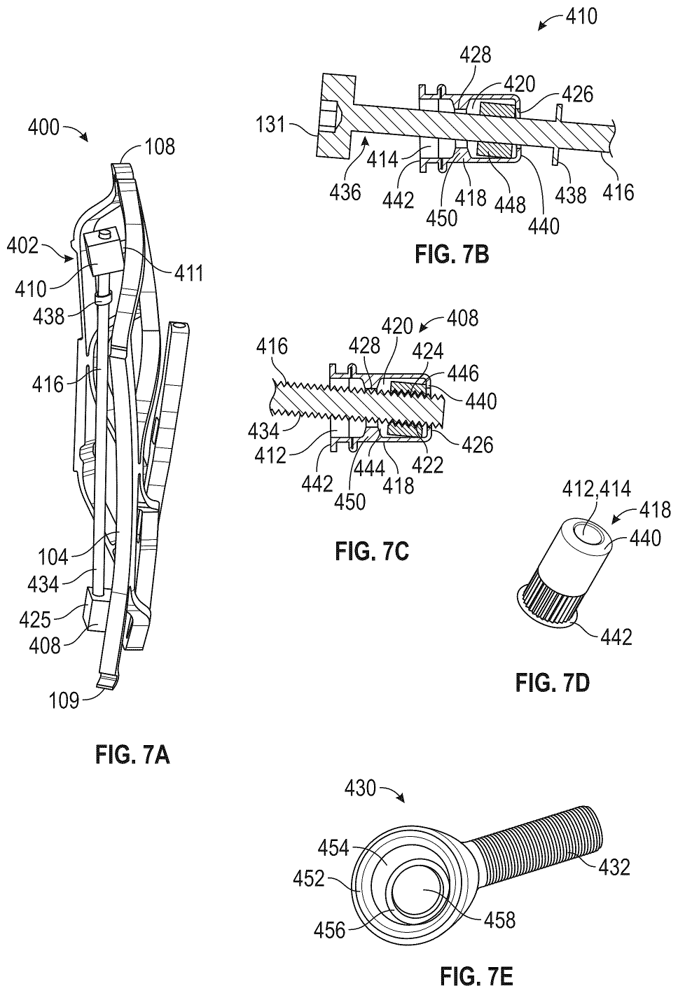

FIGS. 6 and 7A show a section of an exemplary prosthetic heart valve with a frame 400 having an actuation assembly 402 extending radially inwards from the frame 400. As shown in FIG. 6, the frame 400 can have the same or substantially the same basic configuration as frame 102, as previously described, and can function in the same way described above, except for the differences described below. It is to be understood that, while FIGS. 6 and 7A show a frame 400 having an actuation assembly 402 extending radially inwards from the frame 400, in other examples, the actuation assembly 402 can extend radially outwards from the frame 400 instead; that is, the components of the actuation assembly 402 can be coupled to an outer surface of the frame 400. A prosthetic heart valve can comprise the frame 400 and any of the components described above for the prosthetic heart valve 100 (for example, leaflets 158, inner and/or outer skirts, connecting member 125, etc.)

The frame 400 comprises an inflow end 109, an outflow end 108, and a plurality of axially oriented vertical posts 104. Some of the posts 104 can be arranged in pairs of axially aligned first and second posts 404 and 406. The actuation assembly 402 can include a first actuation bracket 408 and a second actuation bracket 410 disposed radially inwards of frame 400, as shown in FIG. 7A. The first and second brackets 408, 410 can be coupled to the inner surface of the frame 400 at axially spaced apart locations. In some examples, such as that illustrated in FIGS. 6 and 7A, the first actuation bracket 408 can be mounted to the first post 404 and the second actuation bracket 410 can be mounted to the second post 406. In some examples, the first bracket 408 can be mounted to the inner surface of an inflow apex 119a and the second bracket 410 can be mounted to the inner surface of an outflow apex 119b.

While FIG. 6 shows only a portion of frame 400 having an actuation assembly 402 positioned radially inwards of the frame 400, it is to be understood that in some examples, frame 400 may comprise a plurality of frame sections identical or substantially identical to that shown in FIG. 6, circumferentially arranged to form an annular frame, similar to the frame 102 as described above and illustrated in FIGS. 1A-2B. In some examples, an actuation assembly 402 can be coupled to each portion of the frame 400. It is to be understood however, that in other examples, one or more of the portions of frame 400 may omit the actuation assembly 402. For example, in a frame 400 comprising six substantially identical portions such as that illustrated in FIG. 2A, an actuation assembly 402 can be coupled to all six sections of the frame 400, or there may be an actuation assembly 402 coupled to a lesser number of frame portions, such as five, four, three, two, or one frame portions.

In the illustrated example, as shown in FIGS. 7B and 7C, the first actuation bracket 408 and the second actuation bracket 410 can include respective bores 412, 414. The bores 412, 414 can be configured to receive other components of the actuation assembly 402, such as an actuator 416, such as in the form of a threaded rod or bolt (also referred to as an “actuator rod” in some examples).

The actuator 416 of the actuation assembly 402 can extend from the first actuation bracket 408 to the second actuation bracket 410 along the longitudinal axis of the frame 400, passing through the first bore 412 and the second bore 414. The actuator 416 can have a first end portion 434 and a second end portion 436. The actuator 416 also includes an externally threaded portion 424.

In one example, as shown in FIG. 7D, the actuation brackets 408, 410 can comprise a cylindrical body 418 with bores 412, 414, respectively. As illustrated in FIG. 7C, a portion of the bore 412 can define a chamber 420 in which a nut 422 may be disposed. The nut 422 has internal threads that engage external threads of an externally threaded portion 424 of the actuator 416 to allow the rotation of the rod to radially expand and radially compress the frame 400, as is discussed in greater detail below. As shown in FIG. 7B, a portion of the bore 414 of the second bracket 410 similarly includes a chamber 420 in which a sleeve 448 may be disposed. The second end portion 436 of the actuator 416 extends through the sleeve 448, which can have a non-threaded inner surface (for example, a smooth cylindrical inner surface) in contact with or facing the outer surface of the second end portion 436 of the actuator 416. In some examples, the second end portion 436 of the actuator 416 can be axially movable and rotatable relative to the sleeve 448.

As shown in FIG. 7C, the nut 422 can be radially and/or axially smaller than the chamber 420, such that the nut 422 may move a limited amount within the cavity such as, for example, when the actuation brackets 408, 410 and/or actuator 416 move during the radial expansion and/or radial compression of the frame 400. For example, the nut 422 has a radial dimension that is smaller than an inner diameter of the chamber 420, which allows the nut 422 to move laterally (perpendicular to a central axis of the chamber 420 and the actuator 416) and pivot or tilt relative to the central axis within the chamber 420. Further, a length of the of the nut 422 in the axial direction can be less than an axial length of the chamber 420 (between an end wall 440 forming aperture 426 and an inner wall 450 forming aperture 428), which allows the nut 422 to move axially within the chamber 420 between the walls 440, 450.

Similarly, the sleeve 448 housed in the second bracket 410 can be radially and/or axially smaller than the chamber 420, such that the sleeve 448 can move a limited amount within the cavity. For example, the sleeve 448 has a radial dimension that is smaller than an inner diameter of the chamber 420, which allows the sleeve 448 to move laterally (perpendicular to a central axis of the chamber 420 and the actuator 416) and pivot or tilt relative to the central axis within the chamber 420. Further, a length of the of the sleeve 448 in the axial direction can be less than an axial length of the chamber 420 (between an end wall 440 forming aperture 426 and an inner wall 450 forming aperture 428), which allows the sleeve 448 to move axially within the chamber 420 between the walls 440, 450.

With continued reference to FIGS. 7B and 7C, the chamber 420 of each of the brackets 408, 410, has a first aperture 426 and a second aperture 428. The apertures 426, 428 receive the actuator 416 and allow the actuator 416 to extend axially through the chambers 420 of the brackets 408, 410. As shown in FIGS. 7B and 7C, the apertures 426, 428 may be larger in diameter than the actuator 416 extending through the brackets 408, 410 such that there are radial gaps between the actuator 416 and the inner surfaces of the apertures 426, 428, yet the apertures 426, 428 are small enough to contain the nut 422 and the sleeve 448 within the respective chambers 420. In this way, the actuator 416 can move (for example, tilt or pivot) a limited amount within the apertures 426, 428 such as, for example, when the actuation brackets 408, 410 and/or actuator 416 move during the radial expansion and/or radial compression of the frame 400.

In some examples, the actuation brackets 408 and 410 can be attached to a frame post or other portion of the frame by a connecting member 411. In some examples, the members 411 may be fixed, such that the actuation brackets 408, 410 remain stationary relative to the frame post or other portion of the frame 400. In such examples, the gaps or spacing between the nut 422 and the sleeve 444 and the inner surfaces of the chambers 420 and the gaps between the actuator 416 and the apertures 426, 428 may allow the actuator 416 to move relative to the actuation brackets 408 and 410 and the frame 400 such that the actuator 416 remains straight or substantially straight when the actuator 416 is actuated to transition the frame 400 from the radially expanded state toward the radially compressed state. In other examples, the connecting members 411 can be hinges or pivot connectors that allow the actuation brackets 408, 410 to pivot relative to the frame 400.

The second end portion 436 of the actuator 416 can include a head portion 131, which is configured to be releasably attached to a delivery system actuator assembly, such as actuator assembly 300 discussed above and shown in FIGS. 4 and 5.

The actuation assembly 402 optionally can also include a stopper 438 disposed along the length of the actuator 416 and positioned between the first actuation bracket 408 and the second actuation bracket 410. Further, the stopper 438 can be integrally formed on or fixedly coupled to the actuator 416, such that it does not move relative to the actuator 416. Thus, the stopper 438 can remain in a fixed axial position on the actuator 416 such that it moves in lockstep with the actuator 416.

Rotation of the actuator 416 in a first direction can cause corresponding axial movement of the first actuation bracket 408 and the second actuation bracket 410 toward one another, thereby causing the inflow end 109 and the outflow end 108 of the frame 400 to move towards one another, decreasing the axial length of and radially expanding frame 400. Specifically, when the actuator 416 is rotated in the first direction, the head portion 131 of the actuator 416 bears against an adjacent surface of the second bracket 410, such as an outflow bracket face 442, while the actuation nut 422 travels along the externally threaded portion 424 of the actuator 416 towards the outflow end 108 until it contacts the inner wall 450 of the first actuation bracket 408. Upon further rotation of the actuator 416 in the first direction, the nut 422 can apply a distally directed force on the first actuation bracket 408 directed towards the outflow end 108 of the frame 400 as the head portion 131 of the actuator 416 applies a proximally directed force on the second actuation bracket 410 directed towards the inflow end 109 of the frame 400. Thus, the rotation of the actuator 416 in the first direction brings the inflow end 109 and the outflow end 108 of the frame 400 closer together, axially compressing and radially expanding the frame 400.

Rotation of the actuator 416 in a second direction opposite to the first direction causes corresponding axial movement of the first and second actuation brackets 408, 410 away from one another, thereby causing the inflow end 109 and the outflow end 108 of the frame 400 to move away from one another, increasing the axial length of and radially expanding frame 400. Specifically, when the actuator 416 is rotated in the second direction, the actuator 416 and the stopper 438 move towards the outflow end 108 of the frame 400 until the stopper 438 abuts the end wall 440 of the second actuation bracket 410, and the nut 422 travels along the threaded portion 424 of the actuator 416 until it abuts the end wall 440 of the chamber 420 of the first actuation bracket 408. Upon further rotation of the actuator 416 in the second direction, the stopper 438 can apply a proximally directed axial force to the inflow end 109 of the second actuation bracket 410 directed towards the outflow end 108 of the frame 400 as the nut 422 applies a distally directed axial force to the first actuation bracket 408 directed towards the inflow end 109 of the frame 400. Thus, the rotation of the actuator 416 in the second direction moves the inflow end 109 and the outflow end 108 of the frame further apart axially expanding and radially compressing the frame 400.

In some examples, the sleeve 448 can be fixed relative to the actuator 416 within the second bracket 410 such that the sleeve 448 and the actuator 416 cannot move axially relative to each other. The sleeve 448 can also be rotationally fixed to the actuator 416 such that they cannot be rotated relative to each, but in other examples, the actuator 416 can be rotatable relative to the sleeve 448. In such cases, the sleeve 448 can assist in the radially expansion and compression of the frame 400. In particular, when the actuator 416 is rotated to radially expand the frame 400, the sleeve 448 can abut the end wall 440, thereby applying a distally directed force to the second bracket 410, while the nut 422 applies a proximally directed force to the first bracket 408, which brings the inflow end 109 of the frame 400 and outflow end 108 of the frame 400 closer together to expand the frame 400. Conversely, when actuator 416 is rotated to radially expand the frame 400, the sleeve 448 can abut the inner wall 450, thereby applying a proximally directed force to the second bracket 410, while the nut 422 applies a distally directed force to the first bracket 408, which causes the inflow end 109 of the frame and outflow end 108 of the frame 400 to move further apart axially to compress the frame 400. In examples where the sleeve 448 is used in this manner, the stopper 438 can be eliminated, or it can be used in conjunction with the sleeve 448 to assist in radially compressing the frame 400.

During the radial expansion and compression of the frame 400, the brackets 408, 410 can pivot or tilt relative to the actuator 416 and the longitudinal axis of the frame 400 to enable the actuator 416 to remain straight throughout the radial expansion and compression of the frame 400. Specifically, when the frame 400 is radially compressed from a radially expanded configuration to a radially compressed configuration, the inflow end 109 and the outflow end 108 may compress to a greater degree than the portions of the frame 400 closer to the axial midpoint, which can cause the opposing end portions of the frame 400 to bend relative to the longitudinal axis of the frame 400. The first bracket 408 can pivot relative to the actuator 416 and the nut 422 from a first orientation (in which a central axis of the bracket 408 is parallel to the longitudinal axis of the frame 400 and the actuator 416) to a second orientation (in which the central axis of the bracket 408 is angled or non-parallel to the longitudinal axis of the frame 400 and the actuator 416 to isolate the bending forces the inflow end 109 of the frame 400 from the actuator 416. Similarly, the second bracket 410 can pivot relative to the actuator 416 and the sleeve 448 from a first orientation (in which a central axis of the bracket 410 is parallel to the longitudinal axis of the frame 400 and the actuator 416) to a second orientation (in which the central axis of the bracket 410 is angled or non-parallel to the longitudinal axis of the frame 400 and the actuator 416 to isolate the bending forces of the outflow end 108 of the frame 400 from the actuator 416. The pivoting movement of the brackets 408, 410 relative to the actuator 416 can thus allow the actuator 416 to remain parallel or substantially parallel to the longitudinal axis of the frame 400, even if the components of frame 400 bend or arch relative to the longitudinal axis of the frame 400.

While FIGS. 7B and 7C show the first and second brackets 408, 410 with a nut 422 disposed in the chamber 420 of the first bracket 408 (that is, with the nut in bracket 408 positioned near the inflow end 109 of the frame 400 section illustrated in FIG. 7A), it is to be understood that in other examples, the positions of the nut 422 and the sleeve 448 can be reversed. For example, the sleeve 448 can be positioned in the first bracket 408 and the nut 422 can be positioned in the second bracket 410. In such examples, the first end portion 434 of the actuator 416 can be unthreaded, and the second end portion 436 of the actuator 416 can be threaded to engage with the nut 422, which can be positioned within the chamber 420 of the second bracket 410. In such examples, the frame 400 can be expanded and compressed in essentially the same manner as described above. Assuming the sleeve 448 is axially fixed relative to the actuator 416, when the actuator 416 is rotated to expand the frame 400, sleeve 448 can apply a proximally directed to the first bracket 408 and the nut 422 can apply a distally directed force to the second bracket 410, which brings the inflow end 109 of the frame 400 and outflow end 108 of the frame 400 closer together to expand the frame 400. Conversely, when the actuator 416 is rotated to compress the frame 400, sleeve 448 can apply a distally directed force to the first bracket 408 and the nut 422 can apply a proximally directed force to the second bracket 410, which causes the inflow end 109 of the frame 400 and outflow end 108 of the frame 400 to move further apart axially to compress the frame 400.

In some examples where the sleeve 448 is in the first bracket 408 and the nut 422 is in the second bracket 410 (such as when the actuator is axially moveable relative to the sleeve 448), the actuation assembly 402 can include two stoppers 438 disposed on the actuator 416, positioned on opposite sides of the first bracket 408 to apply proximally and distally directed forces on the bracket 408 upon rotation of the actuator 416 to produce radial expansion or compression of the frame 400. For example, a distal stopper 438 can be positioned distally of the bracket 408 (and closer to the inflow end 109) and a proximal stopper 438 can be positioned proximally of the bracket 408 (and closer to the outflow end 108).

Rotation of the actuator 416 in a first direction can cause corresponding axial movement of the first actuation bracket 408 and the second actuation bracket 410 toward one another, thereby causing the inflow end 109 and the outflow end 108 of the frame 400 to move towards one another, decreasing the axial length of and radially expanding frame 400. Specifically, when the actuator 416 is rotated in the first direction, the nut 422 travels along the threaded second end portion 436 of the actuator 416 towards the inflow end 109 of the frame 400 until it abuts the end wall 440 of the second bracket 410. The actuator 416 and the stoppers 438 move towards the outflow end 108 of the frame 400 until the distal stopper 438 abuts the end wall 440 of the first bracket 408. Upon further rotation of the actuator 416 in the second direction, the distal stopper 438 can apply an axial force on the first bracket 408 directed towards the outflow end 108 of the frame 400, and the nut 422 can apply an axial force on the second actuation bracket 410 directed towards the inflow end 109 of the frame 400. Thus, the rotation of the actuator 416 in the first direction brings the inflow end 109 and the outflow end 108 of the frame 400 closer together, axially compressing and radially expanding the frame 400.

Rotation of the actuator 416 in a second direction opposite to the first direction causes corresponding axial movement of the first and second actuation brackets 408, 410 away from one another, thereby causing the inflow end 109 and the outflow end 108 of the frame 400 to move away from one another, increasing the axial length of and radially expanding frame 400. Specifically, when the actuator 416 is rotated in the second direction, the nut 422 travels along the threaded second end portion 436 of the actuator 416 towards the outflow end 108 of the frame 400 until it abuts the inner wall 450 of the second bracket 410, while the actuator 416 and the stoppers 438 move towards the inflow end 109 of the frame 416 until the proximal stopper 438 bears against an adjacent surface of the first bracket 408. Upon further rotation of the actuator 416 in the second direction, the nut 422 applies an axial force on the second bracket 410 towards the outflow end 108 of the frame 400, and the proximal stopper 438 applies an axial force on the first bracket 408 towards the inflow end 109 of the frame 400. Thus, the rotation of the actuator 416 in the second direction moves the inflow end 109 and the outflow end 108 of the frame 400 further apart axially expanding and radially compressing the frame 400.

In other examples, the actuation brackets 408 and 410 can be attached to the frame 400 by rotatable coupling members or pivotable coupling members, such as hinges. In such examples, the actuator 416, the nut 422, and the sleeve 448 need not be movable within the chambers 420; that is, the nut 422 and the sleeve 448 can be sized such that they are constrained within the chambers 420 against any movement relative to the brackets 408, 410. When the actuator 416 is rotated to radially compress the frame 400, the brackets 408, 410 can pivot relative to the portions of the frame 400 to which they are attached, thereby decoupling the actuator 416 from the bending forces of the frame 400 and allowing the actuator 416 to remain straight or substantially straight. In other examples, the nut 422, the sleeve 448, and the chambers 420 can be sized as shown in FIGS. 7B and 7C, and the rotatable coupling members (for example, hinges), as well as the gaps between the nut 422 and sleeve 448 and the chambers 420 and the gaps between the actuator 416 and the apertures 426, 428 can allow the actuation brackets 408, 410 and the actuator 416 to move relative to the frame 400 and to each other, thereby decoupling the actuator 416 from the bending forces of the frame 400 and allowing the actuator 416 to remain straight or substantially straight when the frame 400 is compressed.

In some examples shown in FIG. 7E, the frame 400 can, in lieu of the brackets 408, 410, include brackets or mounts in the form of ball joints 430, each of which can be affixed by a connecting member 432 to the frame 400. The connecting member 432 can, in some examples, be an externally threaded rod or bolt that threadedly engages an internally threaded bore or channel in the frame 400. In other examples, the connecting member 432 can be integrally formed as part of the frame 400. The ball joints 430 can be mounted to the inner surface of the frame 400 at axially spaced apart locations. In some examples, the ball joints 430 can be mounted to the first post 404 and the second post 406 (similar to the brackets 408, 410 shown in FIGS. 6 and 7A). In some examples, the ball joints 430 can be mounted to the inner surface of an inflow apex 119a and to the inner surface of an outflow apex 119b. In some examples, the ball joints 430 can be mounted to the posts 404, 406 or the apices 119a, 119b on the outer surface of the frame 400.

The ball joints 430 can be arranged in axially aligned pairs of first and second ball joints 430. In some examples, the first ball joint 430 can be positioned towards the inflow end 109 of the frame 400 and the second ball joint 430 can be positioned towards the outflow end 108 of the frame 400. For example, the first ball joint 430 can be mounted to the first post 404 or to an inflow apex 119a and the second ball joint 430 can be mounted to the second post 406 or an outflow apex 119b. In some examples, each section of the frame 400, such as those depicted in FIGS. 6 and 7A can have an axially aligned pair of ball joints 430 attached. In other examples, only some frame sections can have an axially aligned pair of ball joints 430 attached. For example, in a frame having 6 frame sections, such as that depicted in FIGS. 1A and 2A, each of the 6 frame sections can have an axially aligned pair of ball joints 430 attached, or a lesser number of frame sections, such as 5, 4, 3, 2, or 1 frame sections can have an axially aligned pair of ball joints 430 attached.

The ball joint 430 comprises a socket portion 452 having a spherical socket that receives a sleeve or ball portion 454 having a spherical outer surface. The ball portion 454 is allowed to rotate within the socket portion 452, while remaining axially fixed relative to the frame 400. The ball portion 454 of each ball joint 430 can include a substantially flattened end portion 456 on opposing ends of the ball portion 454, which can provide surfaces for components of the actuation assembly 402, such as the head portion 131 of the actuator 416 or the stoppers 438 to bear against as frame 400 is radially expanded or compressed.

The ball portion 454 has a bore 458 that receives an actuator 416. The inner surface of the bore 458 of one of the ball joints 430 can be threaded to engage external threads of the actuator 416 while the inner surface of the bore 458 of the other ball joint 430 can be non-threaded (smooth) such that rotation of the rotation of the actuator 416 is effective to radially expand and radially compress the frame 400, as is discussed in greater detail below. Thus, the ball portion 454 that has internal threads forms a nut for engaging the actuator 416. For example, the bore 458 of the first ball joint 430 of the axially aligned pair of ball joints (the ball joint 430 positioned closer to the inflow end 109 of the frame 400) can be threaded while the bore 458 of the second ball joint 430 (the ball joint 430 positioned closer to the outflow end 108 of the frame 400) can be non-threaded. In some examples, the bore 458 of the second ball joint 430 of the axially aligned pair of ball joints can be threaded while the bore 548 of the first ball joint 430 can be non-threaded.

The actuator 416 of the actuation assembly 402 can extend through the ball portions 454 of the first and second ball joints 430 along the longitudinal axis of the frame 400. The actuator 416 also includes an externally threaded portion 424, as previously discussed. The externally threaded portion 424 of the actuator 416 engages with the internal threads of the ball portion 454 of the first ball joint 430 to radially expand and/or radially compress the frame 400.

The actuation assembly 402 can also include a stopper 438 disposed along the length of the actuator 416 and positioned between the first and the second ball joints 430 of a pair of ball joints. Further, the stopper 438 can be integrally formed on or fixedly coupled to the actuator 416, such that it does not move relative to the actuator 416. Thus, the stopper 438 can remain in a fixed axial position on the actuator 416 such that it moves in lockstep with the actuator 416. In such examples, rotation of the actuator 416 in the first direction and the second direction radially expands and compresses the frame 400 in substantially the same way as described above for actuation assemblies having the nut 422 in the first bracket 408.

Specifically, when the actuator 416 is rotated in the first direction, the actuator 416 and the stopper 438 can move axially towards the inflow end 109 of the frame 400 until the head portion 131 of the actuator 416 abuts the end portion 456 of the ball portion 454 of the second ball joint 430, and the ball portion 454 of the first ball joint 430 travels along the externally threaded portion 424 of the actuator 416 towards the outflow end 108 of the frame 400, applying a proximally directed axial force on the inflow end 109 of the frame 400 towards the outflow end 108 of the frame 400. Upon further rotation of the actuator 416 in the first direction, the head portion 131 of the actuator 416 can apply a distally directed axial force to the second ball joint 430 directed towards the inflow end 109 of the frame 400, as the movement of the first ball joint 430 along the externally threaded portion 424 of the actuator 416 continues to apply an axially force to the inflow end 109 of the frame 400 directed towards the outflow end 108 of the frame 400. Thus, the inflow end 109 and the outflow end 108 of the frame 400 can be brought closer together, axially foreshortening and radially expanding the frame 400.

When the actuator 416 is rotated in the second direction, the actuator 416 and the stopper 432 can move axially towards the outflow end 108 of the frame 400, until the stopper 432 abuts an end portion 456 of the ball portion 454 of the second ball joint 430, and the ball portion 454 of the first ball joint travels along the externally threaded portion 424 of the actuator 416 towards the inflow end 109 of the frame 400, applying a distally directed axial force on the inflow end of the frame 400 away from the outflow end 108 of the frame 400. Upon further rotation of the actuator 416 in the second direction, the stopper 432 can apply a proximally directed axial force to the second ball joint 430 directed towards the inflow end 109 of the frame 400 as the movement of the first ball joint 430 along the externally threaded portion 424 of the actuator 416 continues to apply a distally directed axial force to the inflow end 109 of the frame 400. Thus, the inflow end and the outflow end of the frame 400 can be pushed further apart, axially extending and radially compressing the frame 400.

As the frame 400 radially compresses, the end portions 108, 109 of the frame 400 may compress further than the axial center of the frame 400, causing the frame 400 to take on an arched or bowed profile relative to the longitudinal axis of the frame 400. Each ball portion 454 of the ball joints 430 can rotate within each corresponding socket portion 452, relative to the longitudinal axis of the frame 400, allowing the bores 458 of each corresponding pair of ball joints 430 substantially aligned, effectively decoupling the bending forces of the frame from the socket portions 454 and the actuator 416. Thus, the actuator 416 extending between each corresponding pair of ball joints 430 can remain straight or substantially straight even if the frame 400 takes on an arched or bowed profile relative to its longitudinal axis.

In one example, in lieu of or in addition to using a stopper 438 on the actuator 416, the portion of the actuator 416 extending through the second ball joint can be axially fixed, but rotatable relative to the ball portion 454. In this manner, when the actuator 416 is rotated to expand the frame, the actuator 416 can apply a distally directed force to the second ball joint 430 and the outflow end 108 of the frame, while the threaded ball portion 454 of the first ball joint 430 applies a proximally directed force to the inflow end 109 of the frame to move the inflow and outflow ends 109, 108 of the frame closer towards one another. Conversely, when the actuator 416 is rotated to compress the frame, the actuator 416 can apply a proximally directed force to the second ball joint 430 and the outflow end 108 of the frame, while the threaded ball portion 454 of the first ball joint applies a distally directed force to the inflow end 109 of the frame to move the inflow and outflow 109, 108 ends of the frame away from one another.

In some examples utilizing ball joints 430, the first ball joint 430 (the one closer to the inflow end 109 of the frame) can have a non-threaded ball portion 454 that engages the actuator and the second ball joint 430 (the one closer to the outflow end 108 of the frame) can have a threaded ball portion 454 that engages external threads of the actuator. In such an example, the actuator can include distal and proximal stoppers configured to apply proximally and distally directed forces to the first ball joint 430 during radially expansion or compression of the frame. In lieu of or in addition to stoppers, the portion of the actuator 416 extending through the non-threaded ball portion 454 of the first ball joint 430 can be rotatable but axially fixed relative to ball portion 454 to apply proximally and distally directed forces to the first ball joint 430 during radially expansion or compression of the frame.

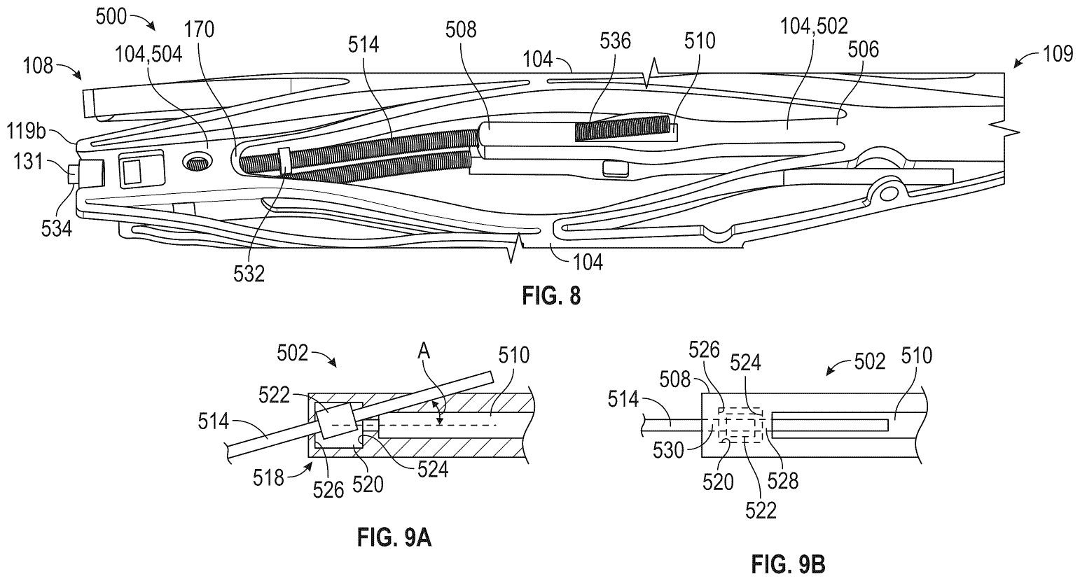

FIG. 8 depicts a segment of some examples prosthetic heart valve frame 500 that minimizes or prevents bending of associated actuators during the crimping and/or radial compression of the prosthetic heart valve. The frame 500 can have the same or substantially the same basic configuration as frame 102, as previously described, and can function in the same way described above, except for the differences described below. A prosthetic heart valve can comprise the frame 500 and any of the components described above for the prosthetic heart valve 100 (for example, leaflets 158, inner and/or outer skirts, connecting member 125, etc.)

With continued reference to FIG. 8, the frame 500 comprises an inflow end 109, an outflow end 108, and a plurality of axially oriented vertical posts 104. Some of the posts 104 can be arranged in pairs of axially aligned first posts 502 and axially aligned second posts 504. As shown in FIG. 8, the first post can have a first end portion 506 (which can be referred to as a fixed end 506) and a second end portion 508 (which can be referred to as a free end 508).

As shown in FIGS. 9A and 9B, the first post 502 can include an axially extending first channel 510 (which can be referred to as a first bore 510). The second post 504 can include an axially extending second channel (not shown) (which can be referred to as a second bore). Together, the first and second channels can allow an actuator 514 to pass through the first post 502 and the second post 504 and extend axially between the first and second posts 502, 504. As shown in FIG. 8, at least a portion the first channel 510 forms an axially extending opening in a surface of the first post 502. In this manner, a portion of the first channel 510 can comprise an axially extending, open slot or groove in the first post 502. This open channel configuration allows a first end portion 516 of the actuator 514 to extend radially outward from the first post 502, for example, when a prosthetic heart valve including the frame 500 is in a radially compressed configuration.

The second end portion 508 of the first post 502 can include a swivel mechanism 518 that allows the actuator 514 to radially rotate relative to a longitudinal axis of the frame 500 during the radial expansion and/or radial compression of the frame. In some examples, such as the one illustrated in FIGS. 9A and 9B, the swivel mechanism 518 can include a nut window or chamber 520 that houses a nut 522.

As shown in FIGS. 9A and 9B, the nut 522 can be radially and/or axially smaller than the nut chamber 520, such that the nut 522 may move a limited amount within the nut chamber 520 such as, for example, when the actuator 514 rotates during the radial expansion and/or compression of the frame 500. For example, the nut 522 has a radial dimension that is smaller than the inner diameter of the nut chamber 520, which allows the nut 522 to pivot or tilt relative to the central axis within the nut chamber 520. Further, a length of the nut 522 in the axial direction can be less than the axial length of the nut chamber 520, which allows the nut 522 to move axially within the nut chamber 520.

In some examples, such as that illustrated in FIG. 9B, the nut chamber 520 can be fully enclosed within the first post 502. In such examples, a first end portion 524 of the nut chamber 520 includes a first aperture 528 and a second end portion 526 of the nut chamber includes a second aperture 530. The apertures 528, 530 allow the actuator 514 to extend axially through the nut chamber 520. In some examples, the apertures 528, 530 can be larger in diameter than the actuator 514. In this way, the actuator 514 can move a limited amount within the apertures 528, 530, such as, for example, when the nut 522 pivots or tilts relative to the central axis within the nut chamber 520 during the radial expansion and/or radial compression of the frame 500.

As shown in FIG. 8, the actuator 514 also can include a stopper 532 (for example, in the form of a nut, washer or flange) disposed thereon. The stopper 532 can be positioned on the actuator 514, between the first post 502 and the second post 504. Further, the stopper 532 can be integrally formed on or fixedly coupled to the actuator 514 such that it does not move relative to the actuator 514. Thus, the stopper 532 can remain in a fixed axial position on the actuator 514 such that it moves in lockstep with the actuator 514.

With continued reference to FIG. 8, the actuator 514 also has a second end portion 534. The second end portion 534 of the actuator 514 can include a head portion 131, which is configured to be releasably attached to a delivery system actuator assembly, such as actuator assembly 300 discussed above and shown in FIGS. 4 and 5.

The actuator 514 can also include an externally threaded portion 536, as shown in FIG. 8. The externally threaded portion 536 of the actuator 514 can engage with an internal thread of the nut 522 to allow the actuator 514 to rotate relative to the nut 522 and cause corresponding axial movement of the first post 502 and the second post 504 toward one another, thereby decreasing the spacing between the first and second posts 502, 504 and radially expanding the frame, while rotation of the actuator 514 in a second direction opposite to the first direction causes corresponding axial movement of the first and second posts 502, 504 away from one another, thereby increasing the spacing between them and radially compressing the frame, as previously described.

While FIG. 8 shows only a portion of frame 500 having an actuator 514 configured to rotate radially relative to the first post 502, it is to be understood that in some examples, frame 500 may comprise a plurality of frame sections identical or substantially identical to that shown in FIG. 8, circumferentially arranged to form an annular frame, similar to the frame 102 as described above and illustrated in FIGS. 1A-2B. In some examples, an actuator 514 can be operatively coupled to each portion of the frame 500. It is to be understood however, that in other examples, one or more of the portions of frame 500 may omit the actuator 514. For example, in a frame 500 comprising six substantially identical portions, such as that illustrated in FIG. 2A, an actuator 514 can be coupled to all six sections of the frame 500, or there may be an actuator coupled to a lesser number of frame portions, such as five, four, three, two, or one frame portions.

When the actuator 514 is rotated in the first direction, the actuator 514 travels axially towards the inflow end 109 of the frame 500 until the head portion 131 of the actuator 514 bears against an adjacent surface of the frame (for example, an outflow apex 119b), while the nut 522 travels axially along the actuator 514 until it contacts the second end portion 526 of the nut chamber 520. Further rotation of the actuator 514 in the first direction causes the nut 522 to bear on the second end portion 526 of the nut chamber 520, applying an axial force directed towards the outflow end 108 of the frame 500 on the first post 502, and the head portion 131 of the actuator 514 to applies an axial force directed towards the inflow end 109 of the frame 500 to the second post 504. Thus, the inflow end 109 and the outflow end 108 of the frame 500 can be pushed closer together, axially foreshortening and radially expanding the frame 500.

When the actuator 514 is rotated in the second direction, the actuator 514 and the stopper 532 move toward the outflow end 108 of the frame until the stopper 532 abuts the inflow end 170 of the second post 504, while the nut 522 travels axially along the actuator 514 towards the inflow end of the frame 500 until it abuts the first end portion 524 of the nut chamber 520. Upon further rotation of the actuator 514 in the second direction, the stopper 532 applies an axial force directed towards the outflow end 108 of the frame 500 to the second post 504, and the nut 522 applies an axial force directed towards the inflow end 109 of the frame 500 to the first post 502. Thus, the inflow end 109 and the outflow end 108 of the frame 500 can be pushed further apart, axially extending and radially compressing the frame 500.

When the frame is in the radially expanded configuration, the actuator 514 and the first post 502 can be parallel or substantially parallel to one another such that an angle A between the actuator and the first post is zero or very small. Thus, the actuator 514 can reside entirely within the channel 510 when the frame is in the radially expanded configuration. As the frame 500 radially compresses from the radially expanded configuration to the radially compressed configuration, the inflow end 109 and the outflow end 108 of the frame 500 may radially compress to a greater degree than the free end 508 of the first post 502. When this occurs, the nut 522 can pivot within the nut chamber 522 as the actuator 514 pivots radially relative to the longitudinal axis of the frame and the angle A increases, allowing the actuator 514 to out of the channel 510, as depicted in FIGS. 8 and 9A. In this manner, the frame 500 can be radially compressed while allowing the actuator 514 to remain straight or substantially straight.

During the radial expansion and compression of the frame 500, the nut 522 within the nut chamber 520 through which the actuator 514 extends can move axially and pivot or tilt radially within the nut chamber. This allows the actuator 514 to pivot radially such that the angle between the actuator 514 and the longitudinal axis of the frame 500 and the angle A between the actuator 514 and the axially oriented first post 502 can change as the frame radially expands and radially compresses. Thus, as the frame 500 takes on an axially bowed or barreled profile, the actuator 514 can remain straight or substantially straight relative to the longitudinal axis of the frame 500.

In this way, the examples previously discussed may provide actuators and actuation assemblies suitable for use with various prosthetic heart valve configurations which may minimize or prevent the bending and/or buckling of the actuators while the valve is in the radially compressed or partially radially compressed configuration during delivery or following the release of the valve from the retention element and/or delivery capsule of the delivery system. Thus, the examples previously discuss can allow for the design of prosthetic heart valves that protect the valve actuators from bending stresses associated with the radial compression of the prosthetic heart valve.

### Delivery Techniques

For implanting a prosthetic valve within the native aortic valve via a transfemoral delivery approach, the prosthetic valve is mounted in a radially compressed state along the distal end portion of a delivery apparatus. The prosthetic valve and the distal end portion of the delivery apparatus are inserted into a femoral artery and are advanced into and through the descending aorta, around the aortic arch, and through the ascending aorta. The prosthetic valve is positioned within the native aortic valve and radially expanded (for example, by inflating a balloon, actuating one or more actuators of the delivery apparatus, or deploying the prosthetic valve from a delivery capsule to allow the prosthetic valve to self-expand). In some examples, a prosthetic valve can be implanted within the native aortic valve in a transapical procedure, whereby the prosthetic valve (on the distal end portion of the delivery apparatus) is introduced into the left ventricle through a surgical opening in the chest and the apex of the heart and the prosthetic valve is positioned within the native aortic valve. In some examples, in a transaortic procedure, a prosthetic valve (on the distal end portion of the delivery apparatus) is introduced into the aorta through a surgical incision in the ascending aorta, such as through a partial J-sternotomy or right parasternal mini-thoracotomy, and then advanced through the ascending aorta toward the native aortic valve.

For implanting a prosthetic valve within the native mitral valve via a transseptal delivery approach, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, into the right atrium, across the atrial septum (through a puncture made in the atrial septum), into the left atrium, and toward the native mitral valve. In some examples, a prosthetic valve can be implanted within the native mitral valve in a transapical procedure, whereby the prosthetic valve (on the distal end portion of the delivery apparatus) is introduced into the left ventricle through a surgical opening in the chest and the apex of the heart and the prosthetic valve is positioned within the native mitral valve.

For implanting a prosthetic valve within the native tricuspid valve, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, and into the right atrium, and the prosthetic valve is positioned within the native tricuspid valve. A similar approach can be used for implanting the prosthetic valve within the native pulmonary valve or the pulmonary artery, except that the prosthetic valve is advanced through the native tricuspid valve into the right ventricle and toward the pulmonary valve/pulmonary artery.

Another delivery approach is a transatrial approach whereby a prosthetic valve (on the distal end portion of the delivery apparatus) is inserted through an incision in the chest and an incision made through an atrial wall (of the right or left atrium) for accessing any of the native heart valves. Atrial delivery can also be made intravascularly, such as from a pulmonary vein. Still another delivery approach is a transventricular approach whereby a prosthetic valve (on the distal end portion of the delivery apparatus) is inserted through an incision in the chest and an incision made through the wall of the right ventricle (typically at or near the base of the heart) for implanting the prosthetic valve within the native tricuspid valve, the native pulmonary valve, or the pulmonary artery.

In all delivery approaches, the delivery apparatus can be advanced over a guidewire and/or an introducer sheath previously inserted into a patient's vasculature. Moreover, the disclosed delivery approaches are not intended to be limited. Any of the prosthetic valves disclosed herein can be implanted using any of various delivery procedures and delivery devices known in the art.

Any of the systems, devices, apparatuses, etc. herein can be sterilized (for example, with heat/thermal, pressure, steam, radiation, and/or chemicals, etc.) to ensure they are safe for use with patients, and any of the methods herein can include sterilization of the associated system, device, apparatus, etc. as one of the steps of the method. Examples of heat/thermal sterilization include steam sterilization and autoclaving. Examples of radiation for use in sterilization include, without limitation, gamma radiation, ultra-violet radiation, and electron beam. Examples of chemicals for use in sterilization include, without limitation, ethylene oxide, hydrogen peroxide, peracetic acid, formaldehyde, and glutaraldehyde. Sterilization with hydrogen peroxide may be accomplished using hydrogen peroxide plasma, for example.

### Additional Examples of the Disclosed Technology

In view of the above-described implementations of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.

Example 1. A prosthetic valve, comprising a radially expandable annular frame having a first end portion, a second end portion, and a longitudinal axis extending between the first end portion and the second end portion, wherein the frame is radially expandable between a radially compressed state and a radially expanded state, a valvular structure disposed within the frame and configured to regulate the flow of blood through the frame in one direction, and an actuation assembly comprising a first actuation bracket and a second actuation bracket coupled to the frame at axially spaced apart locations and an actuator extending axially through the first actuation bracket and the second actuation bracket; wherein the actuator is rotatable relative to the first actuation bracket and the second actuation bracket to radially expand or radially compress the frame; and wherein the actuation assembly decouples bending forces of the frame from the actuator, such that the frame can bend radially outwards from the longitudinal axis independent of the actuator when the frame moves between the radially expanded state and the radially compressed state.

Example 2. The prosthetic valve of any example herein, particularly example 1, wherein a first axial bore extends through the first actuation bracket and a second axial bore extends through the second actuation bracket and the actuator comprises an externally threaded portion that engages an internally threaded nut disposed within a first chamber defined by the first axial bore of the first actuation bracket.

Example 3. The prosthetic valve of any example herein, particularly example 2, wherein an outer diameter of the nut is smaller than an inner diameter of the first chamber, and an axial length of the nut is shorter than an axial length of the first chamber, such that the nut can pivot within the first chamber when the frame is radially expanded or radially compressed.

Example 4. The prosthetic valve of any example herein, particularly example 2, further comprising a stopper mounted on the actuator between the first actuation bracket and the second actuation bracket.

Example 5. The prosthetic valve any example herein, particularly any of examples 2-4, wherein the actuation assembly further comprises a sleeve disposed in a second chamber defined by the second axial bore of the second actuation bracket.

Example 6. The prosthetic valve of any example herein, particularly example 5, wherein the actuator extends through the sleeve.

Example 7. The prosthetic valve of any example herein, particularly example 5, wherein the actuator is integrally formed with the sleeve.

Example 8. The prosthetic valve of any of any example herein, particularly any of examples 5-7, wherein an outer diameter of the sleeve is smaller than an inner diameter of the second chamber, and an axial length of the sleeve is shorter than an axial length of the second chamber, such that the sleeve can pivot within the second chamber when the frame is radially expanded or radially compressed.

Example 9. The prosthetic valve of any example herein, particularly example 1, wherein the first actuation bracket and the second actuation bracket comprise a ball joint comprising a socket, and a sleeve mounted in the socket.

Example 10. The prosthetic valve of any example herein, particularly example 9, wherein the sleeve of the first actuation bracket comprises an internally threaded bore configured to engage with a threaded portion of the actuator and the sleeve of the second actuation bracket comprises a smooth bore configured to admit the actuator.

Example 11. The prosthetic valve of any of any example herein, particularly any of examples 1-10, wherein the actuator comprises a head portion positioned adjacent to the second actuation bracket, and the head portion of the actuator abuts an end portion of the second actuation bracket when the frame is in a radially expanded configuration.

Example 12. The prosthetic valve of any example herein, particularly example 11, wherein the head portion of the actuator is configured to releasably attach to a component of a delivery system.

Example 13. The prosthetic valve of any example herein, particularly any of examples 1-12, wherein the actuator can be rotated in a first rotational direction relative to the first and second actuation brackets to radially expand the prosthetic valve and rotated in a second rotational direction opposite the first rotational direction to radially compress the prosthetic valve.

Example 14. The prosthetic valve of any of any example herein, particularly any of examples 1-13, wherein the first and second actuation brackets are mounted on an interior surface of the frame.

Example 15. The prosthetic valve of any of any example herein, particularly any of examples 1-14, wherein the first and second actuation brackets are mounted on an exterior surface of the frame.

Example 16. The prosthetic valve of any of any example herein, particularly any of examples 1-15, wherein when the prosthetic valve is in a radially compressed configuration, the frame bends radially outwards from the longitudinal axis and the actuator remains straight relative to the longitudinal axis.

Example 17. A prosthetic valve comprising a radially expandable frame; a valvular structure disposed within the frame and configured to regulate the flow of blood through the frame in one direction; and an actuation assembly operatively coupled to the frame; wherein the actuation assembly comprises a first actuation bracket coupled to the frame at a first location, a second actuation bracket coupled to the frame at a second location axially spaced from the first location, an internally threaded nut disposed within the first actuation bracket, and an actuator extending through the nut and from the first actuation bracket to the second actuation bracket; wherein rotating the actuator in a first direction relative to the first and second actuation brackets causes radial expansion of the frame and rotating the actuator in a second direction relative to the first and second actuation brackets causes radial compression of the frame; and wherein the actuation assembly decouples bending forces of the frame from the actuator, such that the frame can bend radially outwards from a longitudinal axis of the frame independent of the actuator upon rotation of the actuator.

Example 18. The prosthetic valve of any example herein, particularly example 17, wherein the actuator comprises an externally threaded portion that engages with a corresponding internal thread of the internally threaded nut.

Example 19. The prosthetic valve of any example herein, particularly any of examples 17-18, wherein the actuation assembly further comprises a stopper mounted to the actuator and positioned between the first actuation bracket and the second actuation bracket.

Example 20. The prosthetic valve of any example herein, particularly any of examples 17-19, wherein the actuation assembly further includes a sleeve disposed around a portion of the actuator and positioned within the second actuation bracket.

Example 21. The prosthetic valve of any example herein, particularly example 20, wherein the sleeve comprises a smooth internal bore through which the actuator extends.

Example 22. The prosthetic valve of any example herein, particularly example 20, wherein the sleeve is integrally formed with the actuator.

Example 23. The prosthetic valve of any example herein, particularly any of examples 17-22, wherein the first actuation bracket and the second actuation bracket each comprise a first aperture and a second aperture that admit the actuator and allow the actuator to extend through the first actuation bracket and the second actuation bracket.

Example 24. The prosthetic valve of any example herein, particularly example 23, wherein the first aperture and the second aperture have an inner diameter sized to form gaps between an outer surface of the actuator and the apertures.

Example 25. The prosthetic valve of any example herein, particularly any of examples 17-24, wherein the actuator comprises a head portion positioned adjacent to an end portion of the second actuation bracket.

Example 26. The prosthetic valve of any example herein, particularly example 25, wherein the head portion of the actuator abuts the end portion of the second actuation bracket when the frame is in a radially expanded configuration.

Example 27. The prosthetic valve of any example herein, particularly any of examples 25-26, wherein the head portion of the actuator releasably attaches to a component of a delivery device.

Example 28. The prosthetic valve of any example herein, particularly any of examples 17-27, wherein the actuator can be rotated in a first rotational direction relative to the first and second actuation brackets to radially expand the frame and rotated in a second rotational direction opposite the first rotational direction to radially compress the frame.

Example 29. The prosthetic valve of any example herein, particularly any of examples 17-28, wherein the frame has a first end portion and a second end portion, and the valvular structure is configured to permit a flow of blood from the first end portion to the second end portion and to prevent a flow of blood from the second end portion to the first end portion.

Example 30. The prosthetic valve of any example herein, particularly example 29, wherein the first actuation bracket is positioned towards the first end portion of the frame and the second actuation bracket is positioned towards the second end portion of the frame.

Example 31. The prosthetic valve of any example herein, particularly example 29, wherein the second actuation bracket is positioned towards the first end portion of the frame and the first actuation bracket is positioned towards the second end portion of the frame.

Example 32. The prosthetic valve of any example herein, particularly any of examples 17-31, wherein the first actuation bracket comprises a first chamber that receives the nut, the first chamber having an inner diameter that is greater than an outer diameter of the nut and an axial length that is greater than an axial length of the nut, such that the nut can pivot within the first chamber when the frame is radially expanded or radially compressed.

Example 33. The prosthetic valve of any example herein, particularly any of examples 20-32, wherein the second actuation bracket comprises a second chamber that receives the sleeve, the second chamber having an inner diameter that is greater than an outer diameter of the sleeve and an axial length that is greater than an axial length of the sleeve, such that the sleeve can pivot within the second chamber when the frame is radially expanded or radially compressed.

Example 34. The prosthetic valve of any example herein, particularly any of examples 17-33, wherein when the frame is in a radially compressed configuration, the frame bends radially outwards from the longitudinal axis and the actuator remains straight relative to the longitudinal axis.

Example 35. A prosthetic valve, comprising a radially expandable frame having a first end portion, a second end portion, and a longitudinal axis extending between the first end portion and the second end portion; a vertically oriented first post parallel to the longitudinal axis, a valvular structure disposed within the frame configured to permit the flow of blood from the first end portion of the frame towards the second end portion of the frame and to prohibit the flow of blood from the second end portion of the frame towards the first end portion of the frame, and an actuation assembly comprising an actuator operatively coupled to the first post; wherein rotating the actuator in a first direction relative to the first post causes radial expansion of the prosthetic valve and rotating the actuator in a second direction relative to the first post causes radial compression of the frame; and wherein the actuator can rotate radially relative to the longitudinal axis such that a first angle between the actuator and the first post can change when the prosthetic valve moves from a radially compressed configuration to a radially expanded configuration or from the radially expanded configuration to the radially compressed configuration.

Example 36. The prosthetic valve of any example herein, particularly example 35, wherein the vertically oriented first post comprises a free end and a fixed end, the free end of the first post comprises a nut chamber that receives an internally threaded nut, the actuator extends through the nut, and wherein the actuator comprises an externally threaded portion that engages internal threads of the nut.

Example 37. The prosthetic valve of any example herein, particularly example 36, wherein the nut is axially and radially smaller than the nut chamber and configured to pivot relative to the longitudinal axis within the nut chamber as the prosthetic valve moves from the radially compressed configuration to the radially expanded configuration or from the radially expanded configuration to the radially compressed configuration.

Example 38. The prosthetic valve of any example herein, particularly any of examples 35-37, wherein the nut chamber comprises a distal end portion and a proximal end portion, and the nut is configured to travel axially along the actuator between the distal end portion and the proximal end portion of the nut chamber.

Example 39. The prosthetic valve of any example herein, particularly any of examples 35-38, wherein the first post comprises a channel that receives an end portion of the actuator.

Example 40. The prosthetic valve of any example herein, particularly example 39, wherein the end portion of the actuator is positioned within the channel when the frame is in a radially expanded configuration and extends radially outwards from the channel when the frame is in a radially compressed configuration.

Example 41. The prosthetic valve of any example herein, particularly any of examples 35-40 wherein the frame comprises a vertically oriented second post axially aligned with the first post and the actuator extends through an axially oriented channel in the second post.

Example 42. The prosthetic valve of any example herein, particularly example 41, wherein the actuation assembly further comprises a stopper mounted to the actuator and positioned between the first post and the second post.

Example 43. The prosthetic valve of any example herein, particularly any of examples 35-42, wherein a proximal end portion of the actuator comprises a head portion that abuts a proximal end portion of the second post when the frame is in a radially expanded configuration.

Example 44. The prosthetic valve of any example herein, particularly example 43, wherein the head portion of the actuator is configured to releasably attach to a component of a delivery apparatus.

Example 45. The prosthetic valve of any example herein, particularly any of examples 35-44, wherein the first angle is substantially zero degrees such that the actuator is substantially parallel to the longitudinal axis when the frame is in the radially expanded configuration.

Example 46. The prosthetic valve of any example herein, particularly any of examples 35-45, wherein the actuation assembly decouples bending forces of the frame from the actuator, such that the frame can bend radially outwards from the longitudinal axis independent of the actuator.

Example 47. A prosthetic heart valve, comprising a radially expandable frame having an inflow end, an outflow end, and a longitudinal axis extending from the inflow end to the outflow end, a valvular structure disposed within the frame and configured to allow the flow of blood through the frame from the inflow end to the outflow end and prevent the flow of blood from the outflow end towards the inflow end, and an actuation assembly operatively coupled to the frame; wherein the actuation assembly comprises a first actuation bracket positioned towards the inflow end of the frame, a second actuation bracket positioned towards the outflow end of the frame, a nut disposed within the second actuation bracket, and an actuator extending from the first actuation bracket to the second actuation bracket and extending through the nut; wherein rotating the actuator in a first direction relative to the first and second actuation brackets causes radial expansion of the frame and rotating the actuator in a second direction relative to the actuation brackets causes radial compression of the frame; and wherein the first and second actuation brackets are configured to pivot relative to the longitudinal axis and the actuator upon rotation of the actuator.

Example 48. The prosthetic heart valve of any example herein, particularly example 47, wherein the actuator comprises an externally threaded portion that engages with corresponding internal threads of the nut.

Example 49. The prosthetic heart valve of any example herein, particularly any of examples 47-48, wherein the actuation assembly further comprises a sleeve positioned in the first actuation bracket and disposed around an end portion of the actuator.

Example 50. The prosthetic heart valve of any example herein, particularly example 49, wherein the sleeve comprises an unthreaded internal bore and the end portion of the actuator extends through the internal bore of the sleeve.

Example 51. The prosthetic heart valve of any example herein, particularly example 49, wherein the sleeve is integrally formed with the actuator.

Example 52. The prosthetic heart valve of any example herein, particularly any of examples 47-51, wherein the actuator comprises a head portion and the head portion of the actuator is positioned further towards the outflow end of the frame than second actuation bracket and abuts an end portion of the second actuation bracket when the frame is in a radially expanded configuration.

Example 53. The prosthetic heart valve of any example herein, particularly example 52, wherein the head portion of the actuator releasably attaches to a component of a delivery device.

Example 54. A prosthetic heart valve comprising a radially expandable frame with a first end portion, a second end portion, a longitudinal axis extending between the first end portion and the second end portion, a vertically oriented first post, and a vertically oriented second post, a valvular structure disposed within the frame and configured to regulate the flow of blood through the frame in one direction, and an actuation assembly comprising an actuator operatively coupled to the frame; wherein the vertically oriented first post comprises a fixed end and a free end, having a nut chamber containing a nut configured to tilt relative to the longitudinal axis within the nut chamber and a slot configured to receive an end portion of the actuator; wherein the actuator extends through the nut and is configured to rotate relative to the longitudinal axis to produce radial expansion of the frame from a radially compressed configuration to a radially expanded configuration or radially compression of the frame from the radially expanded configuration to the radially compressed configuration; and wherein when the frame is in the radially expanded configuration, the end portion of the actuator is disposed within the slot and when the frame is in the radially compressed configuration, the end portion of the actuator can extend radially outwards from the slot.

Example 55. The prosthetic heart valve of any example herein, particularly example 54, wherein the nut chamber comprises a distal end portion and a proximal end portion, and the nut is configured to travel axially between the distal end portion and the proximal end portion of the nut chamber.

Example 56. The prosthetic heart valve of any example herein, particularly any of examples 54-55, wherein the nut chamber is sized to define a radial gap between an outer surface of the nut and an inner surface of the nut chamber.

Example 57. The prosthetic heart valve of any example herein, particularly any of examples 54-56, wherein the actuation assembly further comprises a stopper mounted to the actuator and positioned between the first post and the second post.

Example 58. The prosthetic heart valve of any example herein, particularly any of examples 54-57, wherein a proximal end portion of the actuator comprises a head portion positioned adjacent to an end portion of the second post.

Example 59. The prosthetic heart valve of any example herein, particularly example 58, wherein the head portion of the actuator abuts a proximal end portion of the second post when the frame is in a radially expanded configuration.

Example 60. The prosthetic heart valve of any example herein, particularly any of examples 58-59, wherein the head portion of the actuator is configured to releasably attach to a component of a delivery apparatus.

Example 61. The prosthetic heart valve of any example herein, particularly any of examples 63-74, wherein the actuator is substantially parallel to the longitudinal axis when the frame is in a radially expanded configuration.

Example 62. The prosthetic heart valve of any example herein, particularly any of examples 54-61, wherein the actuation assembly decouples the bending forces of the frame from the actuator, such that the frame can bend radially outwards from the longitudinal axis independent of the actuator.

Example 63. A prosthetic valve, comprising a radially expandable annular frame having a first end portion, a second end portion, and a longitudinal axis extending between the first end portion and the second end portion, wherein the frame is radially expandable between a radially compressed state and a radially expanded state, a valvular structure disposed within the frame and configured to regulate the flow of blood through the frame in one direction, and an actuation assembly comprising a first mount and a second mount coupled to the frame at axially spaced apart locations and an actuator extending axially through the first mount and the second mount; wherein the first mount and second mount each comprise a fixed housing and a sleeve disposed within the fixed housing to form a ball and socket joint, wherein the sleeve is rotatable relative to the fixed housing and the frame; and wherein the actuator is rotatable relative to the first mount and the second mount to radially expand or radially compress the frame.

Example 64. The prosthetic valve of any example herein, particularly example 63, wherein the sleeve of the first mount and the sleeve of the second mount each comprise a bore that admits a component of the actuator.

Example 65. The prosthetic valve of any example herein, particularly example 64, wherein the bore in the sleeve of the first mount is internally threaded and engages a threaded portion of the actuator.

Example 66. The prosthetic valve of any example herein, particularly any of examples 63-65, wherein the bore of the sleeve in the second mount is non-threaded.

Example 67. The prosthetic valve of any example herein, particularly any of examples 63-66, wherein the actuation assembly further includes a stopper mounted on the actuator and positioned between the first mount and the second mount.

Example 68. The prosthetic valve of any example herein, particularly any of examples 63-67, wherein the valvular structure permits the flow of blood from the first end portion of the frame to the second end portion of the frame.

Example 69. The prosthetic valve of any example herein, particularly example 68, wherein the first mount is positioned towards the first end portion of the frame and the second mount is positioned towards the second end portion of the frame.

Example 70. The prosthetic valve of any example herein, particularly example 68, wherein the first mount is positioned towards the second end portion of the frame and the second mount is positioned towards the first end portion of the frame.

Example 71. The prosthetic valve of any example herein, particularly any of examples 63-70, wherein the actuator comprises a head portion positioned adjacent to an end portion of the second mount, and wherein the head portion abuts the end portion of the second mount when the frame is in a radially expanded configuration.

Example 72. The prosthetic valve of any example herein, particularly example 71, wherein the head portion of the actuator is configured to releasably attach to a component of a delivery apparatus.

Example 73. The prosthetic valve of any example herein, particularly any of examples 63-72, wherein when the prosthetic valve is in a radially compressed configuration, the frame bends radially outwards from the longitudinal axis and the actuator remains substantially parallel to the longitudinal axis.

Example 74. A frame or prosthetic heart valve of any preceding example, wherein the frame or prosthetic heart valve is sterilized.

Example 75. A method comprising sterilizing the prosthetic heart valve or the frame of any preceding example.

The features described herein with regard to any example can be combined with other features described in any one or more of the examples, unless otherwise stated. For example, any one or more of the features of one frame or actuator can be combined with any one or more features of another frame or actuator.

In view of the many possible ways in which the principles of the disclosure may be applied, it should be recognized that the illustrated configurations depict examples of the disclosed technology and should not be taken as limiting the scope of the disclosure nor the claims. Rather, the scope of the claimed subject matter is defined by the following claims and their equivalents.

1. A prosthetic valve, comprising:
a radially expandable annular frame having a first end portion, a second end portion, and a longitudinal axis extending between the first end portion and the second end portion, wherein the frame is radially expandable between a radially compressed state and a radially expanded state;
a valvular structure disposed within the frame and configured to regulate the flow of blood through the frame in one direction; and
an actuation assembly comprising a first actuation bracket and a second actuation bracket coupled to the frame at axially spaced apart locations and an actuator extending axially through the first actuation bracket and the second actuation bracket;
wherein the actuator is rotatable relative to the first actuation bracket and the second actuation bracket to radially expand or radially compress the frame; and
wherein the actuation assembly decouples bending forces of the frame from the actuator, such that the frame can bend radially outwards from the longitudinal axis independent of the actuator when the frame moves between the radially expanded state and the radially compressed state.

2. The prosthetic valve of claim 1, wherein a first axial bore extends through the first actuation bracket and a second axial bore extends through the second actuation bracket and the actuator comprises an externally threaded portion that engages an internally threaded nut disposed within a first chamber defined by the first axial bore of the first actuation bracket.

3. The prosthetic valve of claim 2, wherein an outer diameter of the nut is smaller than an inner diameter of the first chamber, and an axial length of the nut is shorter than an axial length of the first chamber, such that the nut can pivot within the first chamber when the frame is radially expanded or radially compressed.

4. The prosthetic valve of claim 2, further comprising a stopper mounted on the actuator between the first actuation bracket and the second actuation bracket.

5. The prosthetic valve of claim 2, wherein the actuation assembly further comprises a sleeve disposed in a second chamber defined by the second axial bore of the second actuation bracket, wherein the actuator extends through the sleeve.

6. The prosthetic valve of claim 5, wherein an outer diameter of the sleeve is smaller than an inner diameter of the second chamber, and an axial length of the sleeve is shorter than an axial length of the second chamber, such that the sleeve can pivot within the second chamber when the frame is radially expanded or radially compressed.

7. The prosthetic valve of claim 1, wherein the first actuation bracket and the second actuation bracket each comprise a ball joint comprising a socket, and a sleeve mounted in the socket.

8. The prosthetic valve of claim 7, wherein the sleeve of the first actuation bracket comprises an internally threaded bore configured to engage with a threaded portion of the actuator and the sleeve of the second actuation bracket comprises a smooth bore configured to admit the actuator.

9. The prosthetic valve of claim 1, wherein the actuator can be rotated in a first rotational direction relative to the first and second actuation brackets to radially expand the prosthetic valve and rotated in a second rotational direction opposite the first rotational direction to radially compress the prosthetic valve.

10. A prosthetic valve comprising:
a radially expandable frame;
a valvular structure disposed within the frame and configured to regulate the flow of blood through the frame in one direction; and
an actuation assembly operatively coupled to the frame;
wherein the actuation assembly comprises a first actuation bracket coupled to the frame at a first location, a second actuation bracket coupled to the frame at a second location axially spaced from the first location, an internally threaded nut disposed within the first actuation bracket, and an actuator extending through the nut and from the first actuation bracket to the second actuation bracket;
wherein rotating the actuator in a first direction relative to the first and second actuation brackets causes radial expansion of the frame and rotating the actuator in a second direction relative to the first and second actuation brackets causes radial compression of the frame; and
wherein the actuation assembly decouples bending forces of the frame from the actuator, such that the frame can bend radially outwards from a longitudinal axis of the frame independent of the actuator upon rotation of the actuator.

11. The prosthetic valve of claim 10, wherein the actuation assembly further comprises a stopper mounted to the actuator and positioned between the first actuation bracket and the second actuation bracket.

12. The prosthetic valve of claim 10, wherein the actuation assembly further includes a sleeve disposed around a portion of the actuator and positioned within the second actuation bracket, wherein the sleeve comprises a smooth internal bore through which the actuator extends.

13. The prosthetic valve of claim 11, wherein the first actuation bracket and the second actuation bracket each comprise a first aperture and a second aperture that admit the actuator and allow the actuator to extend through the first actuation bracket and the second actuation bracket.

14. The prosthetic valve of claim 13, wherein the first aperture and the second aperture have an inner diameter sized to form gaps between an outer surface of the actuator and the apertures.

15. The prosthetic valve of claim 14, wherein a head portion of the actuator abuts an end portion of the second actuation bracket when the frame is in a radially expanded configuration.

16. A prosthetic valve, comprising:
a radially expandable frame having a first end portion, a second end portion, a longitudinal axis extending between the first end portion and the second end portion, and a vertically oriented first post parallel to the longitudinal axis;
a valvular structure disposed within the frame configured to permit the flow of blood from the first end portion of the frame towards the second end portion of the frame and to prohibit the flow of blood from the second end portion of the frame towards the first end portion of the frame; and
an actuation assembly comprising an actuator operatively coupled to the first post;
wherein rotating the actuator in a first direction relative to the first post causes radial expansion of the prosthetic valve and rotating the actuator in a second direction relative to the first post causes radial compression of the frame; and
wherein the actuator can pivot radially relative to the longitudinal axis such that a first angle between the actuator and the first post can change when the prosthetic valve moves from a radially compressed configuration to a radially expanded configuration or from the radially expanded configuration to the radially compressed configuration.

17. The prosthetic valve of claim 16, wherein the vertically oriented first post comprises a free end and a fixed end, the free end of the first post comprises a nut chamber that receives an internally threaded nut, the actuator extends through the nut, and wherein the actuator comprises an externally threaded portion that engages internal threads of the nut.

18. The prosthetic valve of claim 16, wherein the first post comprises a channel that receives an end portion of the actuator, and wherein the end portion of the actuator is positioned within the channel when the frame is in the radially expanded configuration and extends radially outwards from the channel when the frame is in the radially compressed configuration.

19. The prosthetic valve of claim 16, wherein the first angle is substantially zero degrees such that the actuator is substantially parallel to the longitudinal axis when the frame is in the radially expanded configuration.

20. The prosthetic valve of claim 16, wherein the actuation assembly decouples bending forces of the frame from the actuator, such that the frame can bend radially outwards from the longitudinal axis independent of the actuator.
